(19) United States
(12) Patent Application Publication
(10) Pub. No.: US 2025/0346895 A1
(43) Pub. Date: Nov. 13, 2025

(54) GENETIC SEQUENCE-CARBOHYDRATE CONJUGATES FOR ENHANCED LIVER- AND KIDNEY-SPECIFIC TARGETING

(71) Applicant: University of Connecticut, Farmington, CT (US)

(72) Inventors: Raman Bahal, Glastonbury, CT (US); Vikas Kumar, Willimantic, CT (US); Aniket Wahane, Willimantic, CT (US)

(21) Appl. No.: 18/860,452

(22) PCT Filed: Apr. 26, 2023
(86) PCT No.: PCT/US2023/019940

§ 371 Date: Oct. 25, 2024

Related U.S. Application Data
(60) Provisional application No. 63/335,174, filed on Apr. 26, 2022.

(51) Int. Cl. C12N 15/113; A61K 47/54; A61P 1/16

(52) U.S. Cl. CPC C12N 15/113; A61K 47/549; A61P 1/16; C12N 2310/141; C12N 2310/3181; C12N 2310/351; C12N 2320/32

(57) ABSTRACT

A genetic sequence-carbohydrate conjugate is disclosed, having a formula (I): wherein GS is a genetic sequence, preferably a peptide nucleic acid or an oligonucleotide such as an mRNA sequence, an siRNA Sequence, or a DNA sequence, optionally wherein each genetic sequence is natural or modified, and the variables are as described herein. Also disclosed are uses for the genetic sequence-carbohydrate conjugates, including as RNA therapeutics targeting the liver and kidneys of a mammal, including a human.

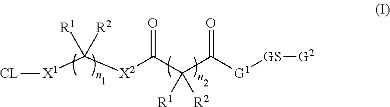

### CROSS-REFERENCE TO RELATED APPLICATION

This application claims priority to, and the benefit of, U.S. Provisional Application No. 63/335,174 filed on Apr. 26, 2022, the entire contents of which are incorporated herein in their entirety.

### BACKGROUND

This application is directed to genetic sequence-carbohydrate conjugates, for example peptide nucleic acid-carbohydrate conjugates, their methods of manufacture, compositions including the conjugates, and their uses. The conjugates are especially useful for improved targeting of liver and kidney cells.

The liver and kidneys are important organs involved in critical body functions including metabolism, detoxification, excretion, synthesis of proteins and lipids, secretion of cytokines and growth factors and immune/inflammatory responses. Liver disorders such as hepatitis, alcoholic or non-alcoholic liver disease, hepatocellular carcinoma, hepatic veno-occlusive disease, and liver fibrosis and cirrhosis are the most common liver diseases. More than 1 in 7 of US adults, or about 37 million people, are estimated to have chronic kidney disease CKD. Renal fibrosis is the final manifestation of chronic kidney disease. Other kidney diseases include cancer, IgA nephropathy, membranous nephropathy, and acute kidney injury.

Many therapeutics are delivered to body tissues that they are not meant to effect, which can result in unintended or harmful side effects. There is accordingly a need for safe and efficient delivery of therapeutic molecules (for example genetic sequences and active agents such as drugs, genes, or proteins) to their target sites in the body including the liver and kidneys. These unmet needs and long unresolved problems are addressed by new compositions and methods described below.

### SUMMARY

A genetic sequence-carbohydrate conjugate is disclosed, having a formula:

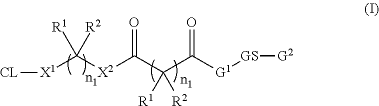

wherein GS is a genetic sequence, preferably a peptide nucleic acid or an oligonucleotide such as an mRNA sequence, an siRNA sequence, or a DNA sequence, optionally wherein each genetic sequence is natural or modified, for example comprises a gamma-serine modified gamma peptide nucleic acid, an alanine gamma peptide nucleic acid, a clamp G-modified peptide nucleic acid, a locked nucleic acid (LNA), a phosphorothioate (PS), a phosphorodiamidate morpholino (PMO), a 2′-O-methyl (2′-O-Me), 2′-O-methoxyethyl (2′-O-MOE), 2′-flouro (2′F), a 5′-methylcytosine, or a combination thereof, the genetic sequence having a 3′ end and a 5′ end, R₁ and R₂ are each independently H or a substituted or unsubstituted C₁ to C₆ alkyl, X¹ is O, NR³, C═O, or C(R³)₂ where R³ is H or a substituted or unsubstituted C₁ to C₆ alkyl, X² is O, NR³, or C(R³)₂ where R³ is H or a substituted or unsubstituted C₁ to C₆ alkyl, G¹ is a direct bond or a group linking the PNA to the conjugate, G² is H or a functional moiety, CL is a carbohydrate ligand comprising 2 to 16 carbohydrate residues derived from a monosaccharide, a disaccharide, a trisaccharide, or a tetrasaccharide, preferably wherein CL comprises a carbohydrate residue derived from a monosaccharide or a disaccharide, optionally wherein the carbohydrate ligand is fully or partially acylated on a hydroxy or amino group thereof with a C₂ to C₁₅ acyl group, preferably wherein the carbohydrate ligand is fully or partially acetylated on a hydroxy or amino group thereof, n¹ is 1 to 20, and n² is 0 to 20.

Methods for the production of the genetic sequence-carbohydrate conjugate, in particular a PNA-carbohydrate conjugate are described.

A pharmaceutical composition comprises the genetic sequence-carbohydrate conjugate, in particular a PNA-carbohydrate conjugate, and a pharmaceutical excipient.

Methods for the use of the genetic sequence-carbohydrate conjugate, in particular a PNA-carbohydrate conjugate are described.

A method for reducing expression of a targeted RNA involved in a health disorder in a subject comprises: providing to a cell of the subject in vivo or ex vivo the genetic sequence-lactobionic acid conjugate as described herein, wherein the binding of the PNA of the conjugate to the targeted RNA reduces expression of the targeted RNA, in particular where the targeted RNA is a microRNA.

A method for targeting DNA and gene editing in a health disorder in a subject comprises: providing to a cell of the subject in vivo or ex vivo a genetic sequence-carbohydrate conjugate according to any one of claims 1 to 20, wherein the DNA of the conjugate targeted to the cell modulates expression of a gene.

### BRIEF DESCRIPTION OF THE DRAWINGS

The following Figures are exemplary embodiments, which are provided to illustrate this disclosure. The Figures are not intended to limit compositions, methods, or articles made in accordance with the disclosure to the materials, conditions, or process parameters set forth herein.

FIG. 1A illustrates a design and synthesis of a lactobionic acid (LBA) appended linker ligand and peptide nucleic acids targeting miR-122. In particular, (A) is a synthesis scheme of lysine linker (1-4) and schematic of LBA conjugation with the lysine linker (5-8). (B) shows the chemical structure of DNA and regular PNA units. (C) shows the nucleotide sequence of mature miR-122-5p (top) and the seed region (underlined). PNAs oligomers to target the seed and full length miR-122 (bottom). PNA 1 and 2 were designed to target the seed region, whereas PNA 3 and 4 can bind to full-length miR-122. PNA 2 and 4 contains succinic acid (SA) at 5′ end for conjugation with the LBA-lysine conjugate. Lysine (K) was added on the 3′ end of the PNA followed by fluorescent probe 5-carboxytetramethylrhodamine (TAM) of each PNA. “000” represents the trioxo-miniPEG linker.

FIG. 2 illustrates conjugation of PNA 4 with LBA and tGalNAc and their quality control assessment. In particular, (A) shows solution-phase conjugation of PNA 4 (14) with LBA (8-16). (B) shows solution-phase conjugation of PNA 4 (14) with tGalNAc (13-18). (C) is a MALDI-MS of ligand conjugated PNAs, PNA4-LBA and PNA4-tGalNAc. The calculated and observed masses of the conjugates are depicted on the respective mass spectra. The inset shows reverse phase-high performance liquid chromatography (RP-HPLC) traces of indicated conjugate.

FIG. 3 illustrates the results of biophysical target binding assessment of PNA-conjugated ligands. (A) shows normalized thermal melting curves of short and full-length PNAs and conjugates with the target DNA sequence of miR-122 under low salt physiological conditions. (B) shows gel shift binding assay at 1:2 DNA to PNA ratio under low salt physiological conditions. The SYBR Gold staining was used to visualize DNA and PNA-DNA heteroduplexes (retarded bands).

FIG. 4 illustrates the results of in vivo biodistribution studies of full length PNA conjugates (PNA 3 and PNA 4). (A) shows IVIS imaging of harvested organs from C57BL6/J mice treated with full-length PNA and ligand conjugates at different time points following 10 mg/kg subcutaneous administration. (B) shows histograms depicting uptake of full-length PNA and ligand conjugates in liver cells from C57BL6/J mice following subcutaneous administration at different time points analyzed by flow cytometry. (C) shows confocal microscopy images of liver cryosections from C57BL6/J mice following subcutaneous administration at 1 h. Blue indicates nucleus where red indicates TAMRA. (D) shows the average radiant efficiency for TAMRA fluorescence in the livers of C57BL6/J mice treated with full-length PNA and ligand conjugates at different time points following subcutaneous administration. Results are represented as mean of n=3 with standard error mean as the error bars.

FIG. 5 illustrates in vivo biodistribution studies of anti-seed PNA conjugates (PNA 1 and PNA 2). (A) shows IVIS imaging of harvested organs from C57BL6/J mice treated with anti-seed PNA and ligand conjugates at different time points following 5 mg/kg subcutaneous administration. (B) shows a histogram depicting uptake of anti-seed PNA and ligand conjugates in liver cells from C57BL6/J mice following subcutaneous administration at 0.5 h and 24 h by flow cytometry. (C) shows Avg. Radiant Efficiency for TAMRA fluorescence in the livers of C57BL6/J mice treated with anti-seed PNA and ligand conjugates at different time points following subcutaneous administration. (D) shows flow cytometry dot plots depicting hepatocyte uptake of anti-seed PNA and ligand conjugates in liver cells from C57BL6/J mice following subcutaneous administration after 1 h. BV786 channel represents hepatocytes stained with ASGPR antibody for ASGPR receptors. FITC channel represents hepatocytes stained with HNF4α antibody for hepatocytes. TAMRA channel represents PNA.

FIG. 6 illustrates (A) relative miR-122, ALDOA and BCKDK (downstream targets of miR-122) expression levels in liver cells of C57BL6/J mice following subcutaneous administration of full-length PNA and ligand conjugates. Results are represented as mean of n=6 with standard error mean as the error bars. Statistical analysis was done using t test. *p<0.05, **p<0.01. (B) shows representative western blots of AldoA and Bckdk proteins (miR-122 downstream targets) in liver cells of C57BL6/J mice following subcutaneous administration of full-length PNA and ligand conjugates. Results are represented as n=2. (C) illustrates relative ALDOA and BCKDK (downstream targets of miR-122) protein levels in liver cells of C57BL6/J mice following subcutaneous administration of full-length PNA and ligand conjugates. Results are represented as mean of n>3 with standard error mean as the error bars. Statistical significance was analyzed on GraphPad Prism software using nonparametric one-way ANOVA. For multiple comparisons, an uncorrected Dunn's test was performed. Significance levels *p<0.05, **p<0.01,***p<0.001 vs indicated treatment groups.

FIG. 7 illustrates (A) H&E staining of liver, kidney and spleen from C57BL6/J mice treated with full-length PNA and ligand conjugates subcutaneously at the end of the efficacy study. (B) shows cytokine panel evaluation in C57BL6/J mice treated with full-length PNA and ligand conjugates subcutaneously at the end of the efficacy study. (C) displays average body weight of C57BL6/J mice during the efficacy study for different treatment groups. Results are represented as mean of n=6 with standard error mean as the error bars. (D) shows blood chemistry analysis including aspartate aminotransferase (AST), alkaline phosphatase (ALP), alanine transaminase (ALT), albumin, globulin, total bilirubin, creatinine, blood urea nitrogen (BUN), creatine phosphokinase (CPK), and electrolytes such as phosphorous, calcium, sodium, potassium, magnesium, chloride, and plasma glucose in C57BL6/J mice at the end of the efficacy study for different treatment groups. Results are represented as mean of n>3 with standard error mean as the error bars.

FIG. 8 illustrates biodistribution of PNA 2-LBA_{Ac} in C57B16/J mice at 5 mg/kg subcutaneous dose. (A) shows IVIS imaging of harvested organs of PNA 1, PNA 2-LBAAc, and saline-treated control mice. (B) shows Avg. Radiant Efficiency for TAMRA fluorescence in the kidneys of C57BL6/J mice. Results are represented as mean of n>2 with standard error mean as the error bars. (C) shows a histogram depicting uptake of PNA and acetylated ligand conjugates in kidney cells from C57BL6/J mice following subcutaneous administration at 24 h and 72 h by flow cytometry. (D) shows confocal microscopy of kidney cryosections from C57BL6/J mice following subcutaneous administration after 24 h. Blue indicates nucleus. Red indicates TAMRA.

FIG. 9 illustrates that LBA_{Ac} ligand efficiently delivered a 22mer long PNA sequence to the kidney. (A) shows the nucleotide sequence of miR-21 and anti-miR-21 PNA and their ligand conjugate. PNA 6 contains succinic acid (SA) at 5′ end for conjugation with LBA-lysine conjugate. Lysine (K) was added on the 3′ end of the PNA followed by fluorescent probe 5-carboxytetramethylrhodamine (TAM) of each PNA. 000 represents the trioxo-miniPEG linker. (B) shows IVIS imaging of harvested organs from C57BL6/J mice treated with PNA and ligand conjugates at 4 h and 24 h time points following 1.5 mg/kg subcutaneous administration. (C) shows a histogram depicting uptake of PNA and acetylated ligand conjugates in kidney cells from C57BL6/J mice following subcutaneous administration at 4 h and 24 h by flow cytometry. (D) shows Avg. Radiant Efficiency for TAMRA fluorescence in the kidneys of C57BL6/J mice. Results are represented as mean of n=2 with standard error mean as the error bars.

### DETAILED DESCRIPTION

Described herein are genetic sequence-carbohydrate conjugates for targeted delivery of the conjugates to specific organs in mammals, in particular humans, and more in particular the liver and the kidneys of a mammal such as a human. In an aspect, the genetic sequence-carbohydrate conjugate is a peptide nucleic acid (PNA)-carbohydrate conjugate (PNAC). In particular, the conjugate is a molecule including a carbohydrate ligand covalently linked to a genetic sequence via linker backbone covalently bonded to both. The carbohydrate ligand can be selected to target the liver or kidney. Preferably the conjugate selectively binds to specific receptors on cells to deliver the genetic sequence or other therapeutic agent to the cells bearing the receptors.

The conjugates used herein include a genetic sequence (GS) having a 3′ end and a 5′end, which can be a PNA or an oligonucleotide such as an mRNA sequence, an siRNA sequence, or a DNA sequence. Each genetic sequence can be natural or optionally modified, for example in an order of nucleotides or via modifications as a gamma-serine modified gamma peptide nucleic acid, an alanine gamma peptide nucleic acid, a clamp G-modified peptide nucleic acid, a locked nucleic acid (LNA), a phosphorothioates (PS), a phosphorodiamidate morpholino (PMO), a 2′-O-methyl (2′-O-Me), 2′-O-methoxyethyl (2′-O-MOE), 2′-flouro (2′F), a 5′-methylcytosine, or a combination thereof. In an aspect, the genetic sequence is a PNA. The PNA can be modified as described below,

In an aspect the conjugates bear 1 to 8, or 1 to 5, or 2 to 5, or 2 to 4 carbohydrate residues (ligands). The number and type of carbohydrate ligands are selected to target the liver or kidneys, preferably to selectively target the liver and kidneys. For example, the carbohydrate ligand can be selected to target the Asialglycoprotein receptor (ASPGR) expressed on cells. ASGPR is a C-type lectin, primary expressed on the sinusoidal surface of hepatocytes. In an aspect the conjugates bear 1 to 8, or 1 to 5, or 2 to 5, or 2 to 4 galactose ligands to target the ASPGR on liver and kidney cells. In an aspect the conjugates bear 1 to 8, or 1 to 5, or 2 to 5, or 2 to 4 galactose amine (GalNAc) ligands to target the ASPGR on liver and kidney cells. In another aspect the conjugates bear 1 to 8, or 1 to 5, or 2 to 5, or 2 to 4, or 2 to 3 lactobionic acid ligands to target the ASPGR on liver and kidney cells.

The carbohydrate ligand(s) of the conjugate can be fully or partially acylated on a hydroxy or amino group thereof with a C2 to C15 acyl group. For example, the carbohydrate ligand can be fully or partially acetylated on a hydroxy or amino group. The acetylation of a carbohydrate ligand can be performed using an acetylating reagent such as acetic anhydride, acetyl chloride, mixed anhydrides, acids with coupling agents such as DCC or like reagents and a base such as triethyl amine, pyridine, DIEA, DMAP or the like, or an organic, inorganic, or polymeric base as used in the art. In an aspect, the carbohydrate ligand is a GalNAc residue that is fully or partially acylated, preferably acetylated, preferably fully acetylated. Alternatively, the carbohydrate ligand is a lactobionic acid residue that is fully or partially acylated, preferably acetylated, preferably fully acetylated.

The carbohydrate ligands can be covalently attached to the genetic sequence by a backbone linker as shown in Formula I. A variety of backbones can be used, but in general contain at least two functional groups, for example at least two amino groups, one or more for reaction with the carbohydrate ligand(s) and one or more for reaction with the genetic sequence. The amino groups can be selectively protected as known in the art and as described in the Examples. The backbone can include moieties to modify properties such as solubility. For example, lysine and arginine residues can be present in a backbone.

In an aspect as shown in Formula I, a group G¹ or G² can be optionally present. G¹ can be a linker from the backbone to the genetic sequence, for example a linker having 1 to 20 carbon atoms, and optionally one or more reactive groups such as hydroxy, carboxy, thio, or amino. In another aspect, G¹ or G² can be a functional moiety. The functional moiety G¹, G² can provide a structural feature to the conjugates that can impart a desired function such as stearic separation from a binding ligand, enhancing hydrophilicity or hydrophobicity, facilitating absorption, of the conjugates, facilitating distribution of the conjugate in the body, or other functions advantageous in medicinal chemistry and drug design. The functional moiety can be linked between the backbone and the genetic sequence or at a terminal end of the genetic sequence, or both. In an aspect, a functional moiety G¹, G² is, for example, a residue of a polyethylene glycol, a polypropylene glycol, or a polyethylene-propylene glycol. In an aspect, G¹ or G², or both can be polyethylene glycol (PEG) group. The PEG group can contain 1 to 25 ethylene glycol residues (—OCH₂CH₂O—) that can terminate in a free hydroxy, amino, ether, or like functional moiety. The which is optionally bonded to a ligand, a backbone or structure of a conjugate.

In another aspect, the functional moiety can include a therapeutic agent. For example, kielin, tolvaptan, nintedanib, paclitaxel, bleomycin, cyclosporin, cisplatin, romidepsin, doxorubicin, docetaxel, danunorubicin, vincristine, methotrexate, cyclophosphamide, venetoclax, hydroxyurea, mercaptopurine, prednisolone, cytarabine, or pirfenidone. Other therapeutic agents can be found in the Merck Index published by the Royal Society of Chemistry published in print and online at https://www.rsc.org/merck-index. For example, G¹ can be a linker between the backbone and the genetic sequence, and include a therapeutic agent covalently bound thereto. Alternatively, or in addition, the group G² can be a therapeutic agent covalently bound to the genetic sequence either directly or by a linker. Although not shown in Formula I, it is also possible for a functional moiety such as a therapeutic agent to be linked to the backbone using a linkage similar to that linking the carbohydrate residue.

In an aspect, the conjugate is a genetic sequence-lactobionic acid conjugate. Lactobionic acid (LBA) is a disaccharide formed from gluconic acid and galactose. In some embodiments, lactobionic acid is derivatized as part of a conjugate. PNA-lactobionic acid conjugate of Formula Ia

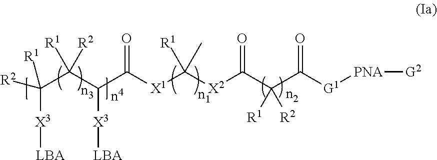

wherein LBA is a lactobionic acid residue, X¹ is NR³, O, or C(R³)₂ where R³ is H or a substituted or unsubstituted C₁ to C₆ alkyl, each X³ is independently O, NR³, or C(R³)₂ where R³ is H or a substituted or unsubstituted C₁ to C₆ alkyl, n₃ is 0 to 20, and n₄ is 1 to 8. Preferably, G¹ is a group linking the PNA to the conjugate, R¹ and R² are each H, X¹, X², and X³ are each NH, and n₁=6, n₂=2, and n₃=4. In an aspect, R¹ and R² are each H, n₃=4, and the PNA is linked at the 5′ end to the conjugate.

For example, the PNA-lactobionic acid conjugate can be of formula Ia-1

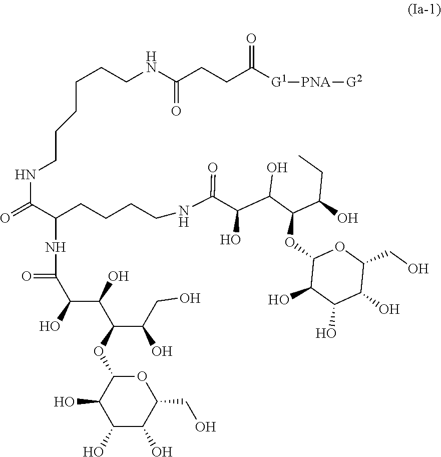

wherein G¹ and G² are as defined above, preferably wherein G¹ is a functional moiety linking the PNA to the conjugate and G² is a functional moiety. Optionally in any of the Formulas Ia and Ia-1, the hydroxyl groups can be fully or partially acylated with an acyl group having from 2 to 15 carbon atoms or 2 to 8 carbon atoms, preferably acetylated, more preferably fully acetylated as described above.

In another aspect, the conjugate can be of Formula Ib

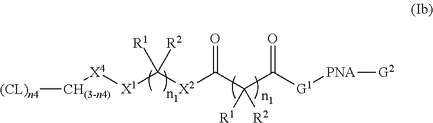

wherein X¹ is C═O or C(R³)₂ where R³ is H or a substituted or unsubstituted C₁ to C₆ alkyl, X⁴ is O, NR³, or C(R³)₂ where R³ is H or a substituted or unsubstituted C₁ to C₆ alkyl, CL is a carbohydrate residue linked to CH by a 1 to 30 atom linker chain comprising a substituted or unsubstituted C₁ to C₁₂ alkyl or a C₆ to C₁₂ aryl comprising an amide, ester, or ether group, and n₄ is 2 or 3. The carbohydrate residue in Formula Ib can be derived from N-acetylgalactosamine, and can be a fully or partially acylated carbohydrate residue wherein the acyl groups have 2 to 15 carbon atoms or 2 to 8 carbon atoms, for example a fully or partially acetylated carbohydrate residue, such as fully acetylated.

A method of conjugating a genetic sequence to a carbohydrate ligand to provide the genetic sequence-carbohydrate conjugate is described. The method includes functionalizing the genetic sequence to provide free —COOH functionality; and forming a bond between the free —COOH functionality of modified genetic sequence and Y² of a compound of a formula II

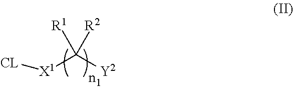

wherein Y² is —NHR³ or —OH. The method can be performed by solution-phase or solid-phase synthesis or a combination thereof. The genetic sequence, for example a PNA, can be obtained by solution- or solid-phase synthesis as is known in the art, or a combination thereof. It can be modified as described below. In addition, the method can further include modifying the genetic sequence with a precursor of G¹, G², or a combination thereof, before functionalizing the genetic sequence.

In an aspect, a method of conjugating a genetic sequence to a lactobionic acid-backbone ligand to provide a genetic sequence-lactobionic acid conjugate includes functionalizing the genetic sequence to provide free —COOH functionality; and forming a bond between the free —COOH functionality of modified genetic sequence and Y² of a formula III

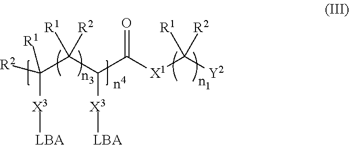

wherein Y² is —NHR³ or —OH. Again, the genetic sequence is preferably a PNA. In an aspect, the method can further comprise reacting lactobionic acid with a backbone of a formula IV

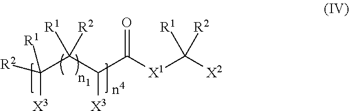

wherein X³ is an —OH or NHR³, and X² is a protected 0 or protected NHR³.

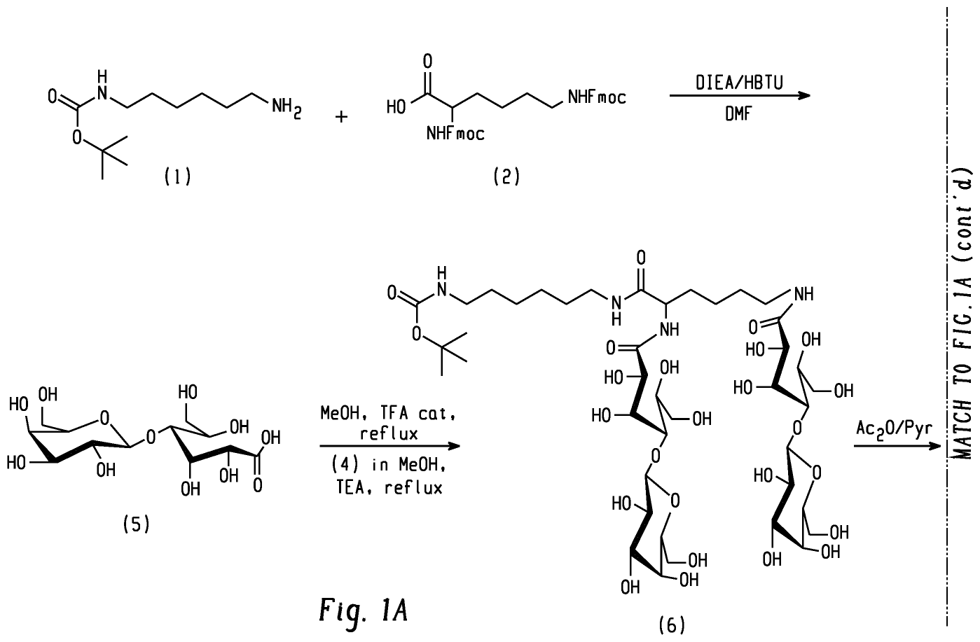

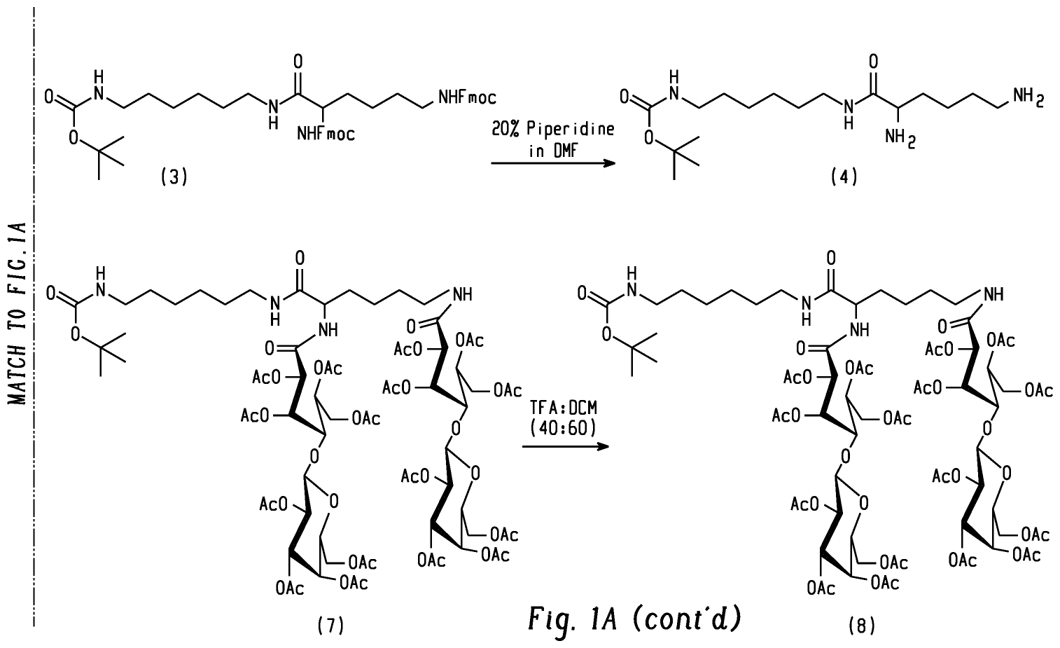

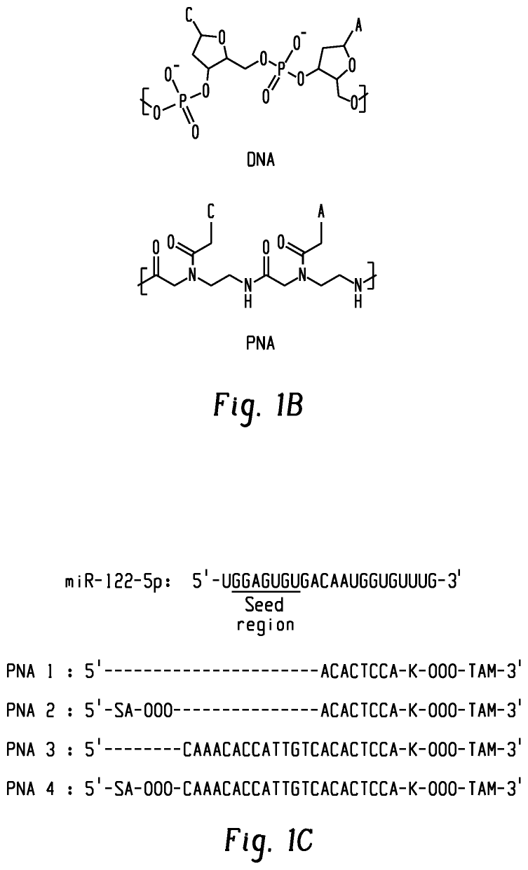

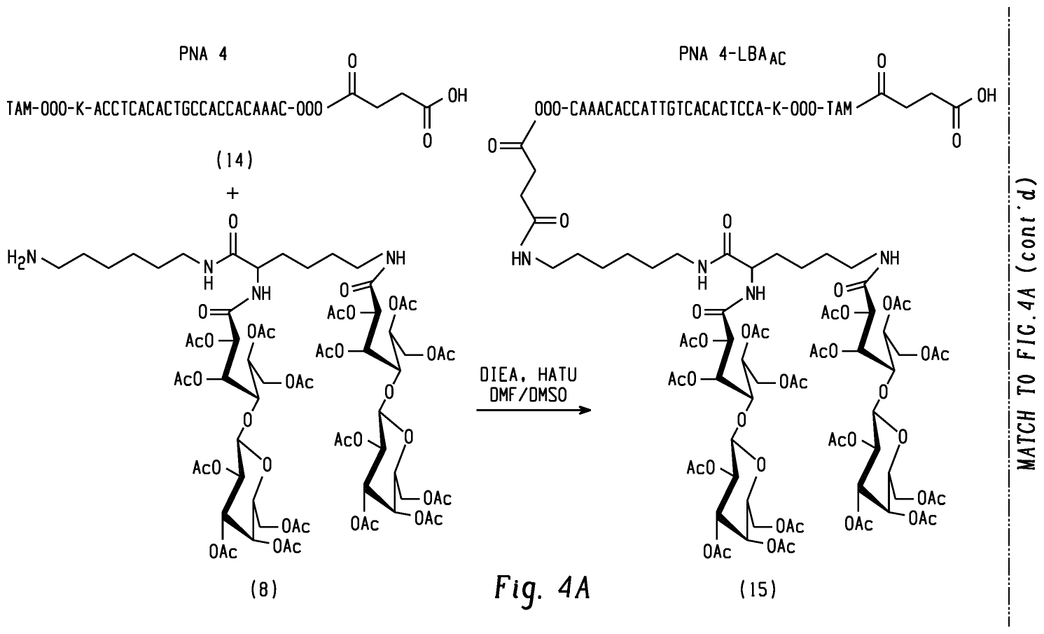

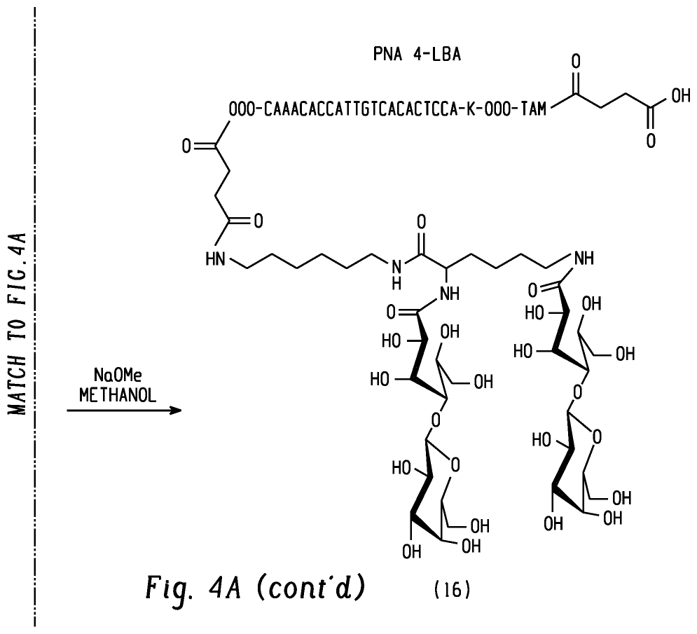

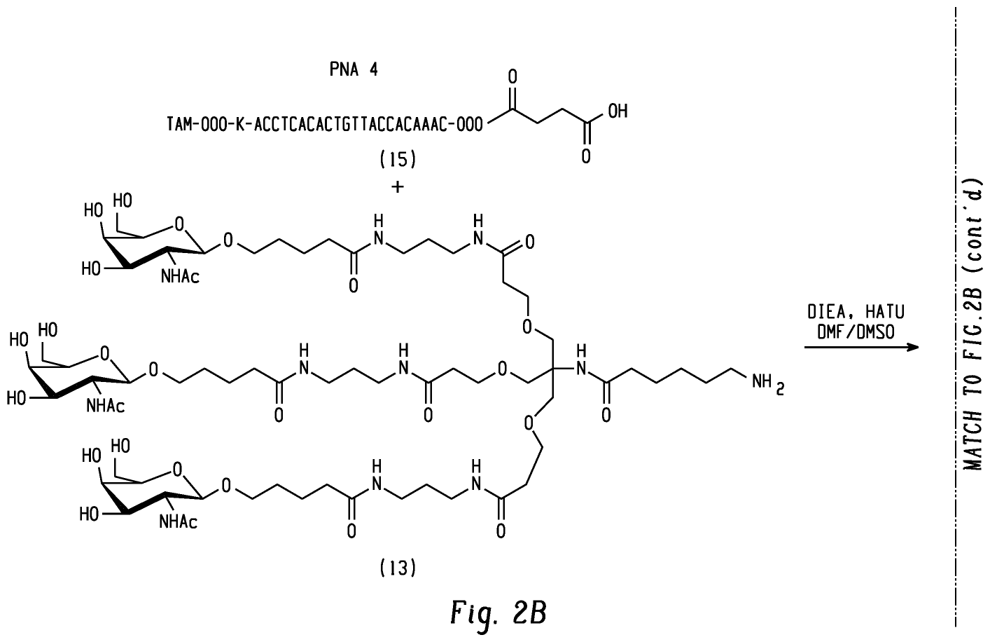

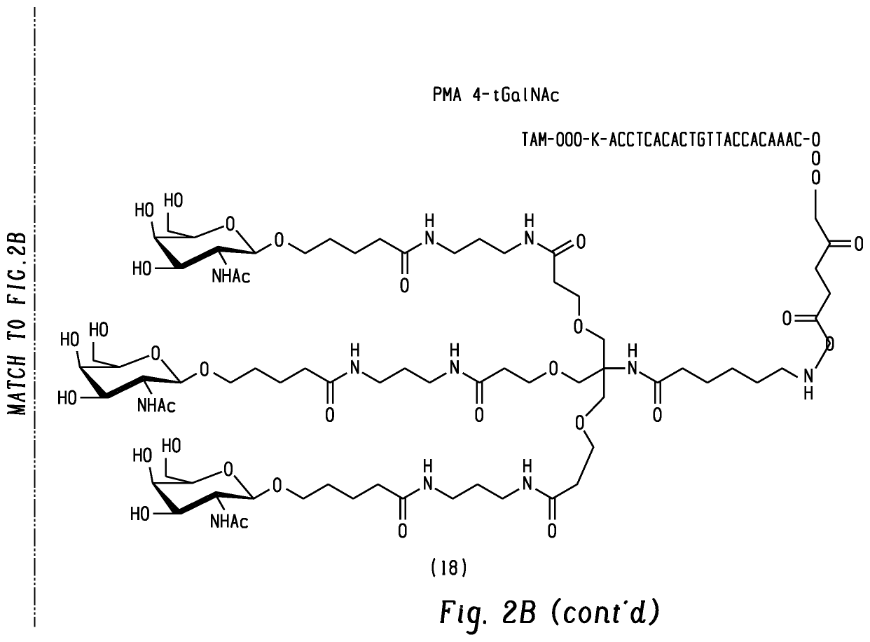

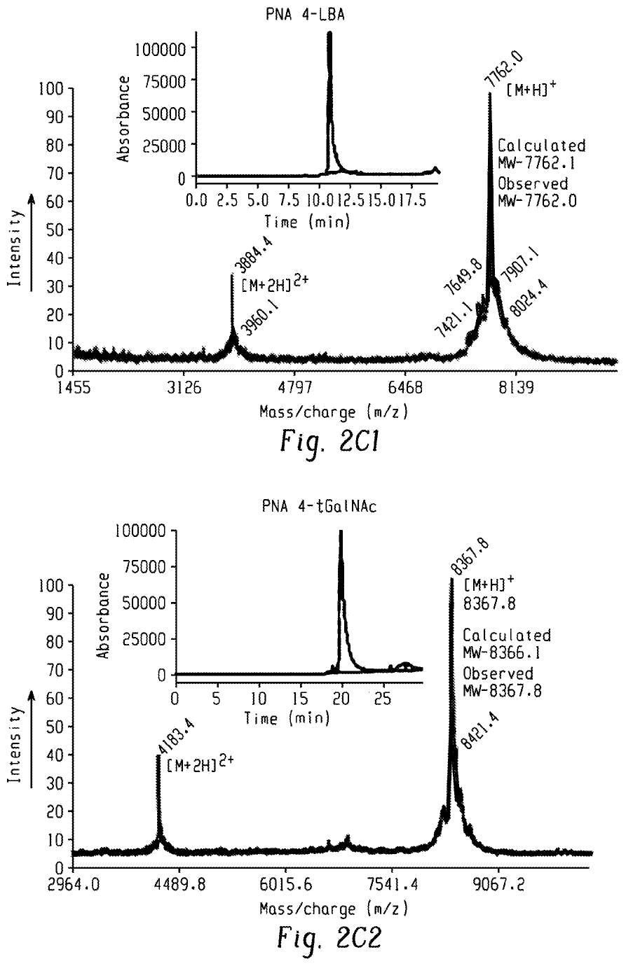

In an aspect, as described in the Examples and shown in FIGS. 1 and 2, a lactobionic acid residue can be coupled to a backbone comprising a lysine residue by its alpha and epsilon amino groups. The lysine carboxyl group is in turn coupled to an amino group on an alkyl diamine, and the other amino group is coupled to a succinyl COOH group linked to a peptide nucleic acid. In other aspects, the alkyl diamine can be substituted by an alkane diol to form a backbone with ester linkages. Alternatively, the succinic acid at the 5′ end can be replaced by a substituted or unsubstituted C to C₂₀ dicarboxylic acid. A PNA is modified with a functional moiety for example a trioxo-miniPEG spacer and succinic acid at the 5′ end to provide a free —COOH functionality after cleavage. Some PNAs so modified are commercially available. The free COOH group can then be reacted with an amino group, hydroxy group, alkyl halide, or other suitable functional group on a ligand backbone, for example lactobionic acid or GalNAc.

Particularly when GalNAc is used, the GalNAcs can be linked to the backbone by groups bearing an alkyl ether, amide, ester residues to provide the carbohydrate ligand. Some of these ligands are available commercially or can be synthesized using chemical synthesis methods familiar to one of ordinary skill in the art. General methods for chemical synthesis may be found in, among other sources, “Comprehensive Organic Transformations: A Guide to Functional Group Preparations,” Richard C. Larock, Wiley-VCH: 1999 and in “March's Advanced Organic Chemistry: Reactions, Mechanisms and Structure”, Jerry March & Michael Smith, John Wiley & Sons Inc.: 2001. Of course, other carbohydrate residues can be similarly linked to the backbone by a group, e.g., a chain, bearing alkyl ether, amide, or ester residues to form the ligand.

Methods for use of the conjugates, are further described. For example, the conjugates can be used to treat cancers in the liver and kidneys. In an aspect the conjugates can be used to treat renal fibrosis. In another aspect, the conjugates can be used to treat renal cancer. In another aspect a conjugate can be used to treat kidney disease. The formulations can be administered directly to a subject for in vivo gene therapy.

The conjugates, in particular the PNACs, can be used as an RNA therapeutic agent. In particular, the conjugates in particular the PNACs, can target microRNA (miRNA) sequences. The conjugates can be used to control gene expression at the post-transcription level. miRNAs play key roles in maintaining physiological processes by controlling gene expression through regulating messenger RNA (mRNA) stability and translation. Use of the conjugates to target an RNA in a cell, such as an mRNA or miRNA, can inhibit expression of the RNA at the translational stage in the case of mRNA, and/or affect gene expression by downregulation or upregulating expression of the miRNA and its downstream effects on its target genes. The conjugates can be used to control aberrant expression of miRNAs causing several devastating diseases. The conjugates can be used to treat cancers wherein, atypical miRNA levels lead to altered processes, including differentiation, proliferation, and apoptosis. In a preferred embodiment, the conjugates are used to treat cancers in the liver and kidneys. In an aspect a conjugate can be used to treat renal fibrosis. In an aspect a conjugate can be used to treat renal cancer. In an aspect a conjugate can be used to treat kidney disease.

Accordingly, in an aspect, a method for reducing expression of a targeted RNA involved in a health disorder in a subject comprises providing to a cell of the subject in vivo or ex vivo the genetic sequence-lactobionic acid conjugate as described herein, wherein the binding of the PNA of the conjugate to the targeted RNA reduces expression of the targeted RNA, in particular, the targeted RNA is a microRNA. In an aspect, the RNA therapeutics are used in targeting liver or kidney cells, or a combination thereof to regulate expression of cellular nucleic acid function of a subject in need thereof, in particular cancer cells, including liver or kidney cancer cells or a combination thereof. In an aspect, the PNA comprises a kidney-specific microRNA, still more specifically miR-21. The condition (need) for treatment can be renal fibrosis and polycystic kidney disease.

In another aspect, a method for targeting DNA and gene editing in a health disorder in a subject comprises: providing to a cell of the subject in vivo or ex vivo a genetic sequence-carbohydrate conjugate according to any one of claims 1 to 20, wherein the DNA of the conjugate targeted to the cell modulates expression of a gene.

The genetic sequence-carbohydrate conjugate can be used for treatment of a subject in need thereof ex vivo or in vivo. The methods typically include contacting a cell ex vivo or in vivo with an effective amount of a conjugate, optionally in combination with a potentiating agent, to deliver a therapeutic agent, for example to modify the expression of an RNA. In an aspect, the method includes contacting a population of target cells with an effective amount of the conjugate, to modify the expression of RNA to achieve a therapeutic result.

The genetic sequence-carbohydrate conjugate is generally provided as a formulation including include an effective amount of a conjugate and a polymer, lipid, protein, or other pharmaceutical excipient for the organ-specific delivery. Pharmaceutically acceptable carrier (also referred to as an excipient in the art), where the formulation is selected to suit the mode of administration. Pharmaceutically acceptable carriers are determined in part by the particular conjugate being administered, as well as by the particular method used to administer the conjugate. For example, the formulations may be for administration topically, locally, or systemically in a suitable pharmaceutical carrier. Accordingly, there is a wide variety of suitable formulations for the conjugates. Remington's Pharmaceutical Sciences, 15th Edition by E. W. Martin (Mark Publishing Company, 1975), discloses typical carriers and methods of preparation. For example, the formulations can include pharmaceutically acceptable carriers such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. The conjugates can also be encapsulated in suitable biocompatible microcapsules, microparticles, nanoparticles, or microspheres formed of biodegradable or non-biodegradable polymers or proteins or liposomes for targeting to cells. The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation. Such systems are well known to those skilled in the art and may be optimized for use with the appropriate nucleic acid.

Formulations suitable for parenteral administration, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The conjugates may take such forms as sterile aqueous or nonaqueous solutions, suspensions, and emulsions, which can be isotonic with the blood of the subject in certain aspects. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions, or suspensions, including saline and buffered media. Parenteral vehicles include sodium chloride solution, 1,3-butanediol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents, and inert gases. In addition, sterile, fixed oils are conventionally employed as a solvent or suspending medium. For this purpose, any bland fixed oil including synthetic mono- or di-glycerides may be employed. In addition, fatty acids such as oleic acid may be used in the preparation of injectables. Those of skill in the art can readily determine the various parameters for preparing and formulating the conjugates without resort to undue experimentation.

The conjugates, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

An effective amount or therapeutically effective amount of the conjugate can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder. The precise dosage will vary according to a variety of factors such as formulation and subject-dependent variables (e.g., age, immune system health, clinical symptoms etc.).

The conjugates, in particular a formulation including the conjugate, can be administered to or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

### EXAMPLES

This disclosure is illustrated by the following Examples, which are not intended to limit the claims.

Except where otherwise specified, materials and reagents were obtained from Sigma-Aldrich or Thermo Fisher Scientific and used as received. The tGalNAc ligand was purchased from Sussex Research, Ottawa, Canada.

### Example 1. Synthesis of PNA-Carbohydrate Conjugates

The PNA-LBA conjugates were synthesized as shown in FIGS. 1A and 1n FIGS. 2A and 2B.

### 1,6-Diamino Hexane-Lysine Backbone:

The 1,6-diamino hexane-lysine backbone is synthesized by an amide coupling reaction (FIG. 1A). The synthesis began by coupling FMOC-protected lysine with 6-amino Boc-protected 1,6-diamino hexane in DMF solvent with HBTU and DIEA. The two FMOC groups on lysine were then deprotected using a 20% piperidine in DMF solution to free the alpha and epsilon amino groups on lysine to provide the backbone for coupling to two lactobionic acid molecules.

### Carbohydrate Ligand: LBA-Backbone Ligand

In the second step shown in FIG. 1A, the backbone was functionalized with LBA. LBA was first converted into lactone form by refluxing in methanol with a catalytical amount of TFA and then the solution was basified with DIPEA for reaction of the lactone form to the backbone. Following reaction, hydroxyl groups on LBA were acetylated for purification. The identity of the reaction products was confirmed by mass spectroscopy and by ¹H and ¹³C NMR spectroscopy.

### Functionalized Peptide Nucleic Acid:

PNA oligomers targeting miR-122 (FIG. 1C) were synthesized on an MBHA resin solid support implementing known and standard BOC deprotection and synthesis protocol. Short (8mer) and full-length (22mer) PNAs targeting the seed region and complete sequence of miR-122 were synthesized using the regular PNA monomers.

Lysine was added to the 3′ end of the PNA using standard methods. For solution-phase conjugation, the PNA oligomers were then modified with a trioxo-miniPEG™ spacer at the 3′ end (using 11-(boc-amino)-3,6,9-trioxaundecanoic acid), and succinic acid at the 5′ end to produce a free —COOH functionality after cleavage. Solid phase synthesis was carried out using 0.2 M solution of PNA monomer/lysine/trioxo-miniPEG/succinic acid/TAMRA in NMP, 0.52M DIEA in DMF, and 0.39M HBTU in DMF. TAMRA fluorescence dye was coupled on the 3′ end of PNAs through the trioxo-miniPEG spacer for cellular uptake and biodistribution studies. Detailed procedures for these materials and methods for PNA synthesis on MBHA resin was reported previously (Malik, Shipra, Frank J. Slack, and Raman Bahal. “Formulation of PLGA nanoparticles containing short cationic peptide nucleic acids.” MethodsX 7 (2020). 101115).

### Carbohydrate-PNA Conjugate:

As shown in FIG. 2A, the functionalized PNA (PNA 4) was coupled to the amino group of the LBA-backbone ligand via the succinic acid at the 5′ end. Reaction was carried out in a DMF/DMSO solvent mixture using HATU as coupling reagent with DIEA as base. The obtained amide bearing acetyl protection on the hydroxy groups of LBA was treated with sodium methoxide in methanol to obtain the PNA 4-LBA conjugate.

As shown in FIG. 2B, the GalNAc-PNA 4 conjugates were similarly prepared by reaction of the functionalized PNA containing a trioxo-miniPEG spacer and a succinic acid at the 5′ end with a free —COOH with the amino group of the GalNAc linked backbone using HATU as the coupling reagent, catalyzed by DIEA in DMF/DMSO solvent.

The HPLC purity profile and MALDI mass spectra of the carbohydrate ligand conjugated PNAs were recorded at every step for quality control purposes (FIG. 2C). The PNAs, PNA4-LBA and PNA4-tGalNAc products were analyzed by MALDI-MS indicating product formation. The products were also analyzed by reverse phase HPLC as shown in the inset in FIG. 2C.

### Example 2. In Vitro Binding Studies

Modification on the PNA strand should not hinder its target binding. To address whether modification of the PNA strand by conjugation would hinder target binding, PNAs and their carbohydrate-PNA conjugates were evaluated for their affinity for their target by gel shift assay and thermal melting curve analysis.

### Gel-Shift Assay:

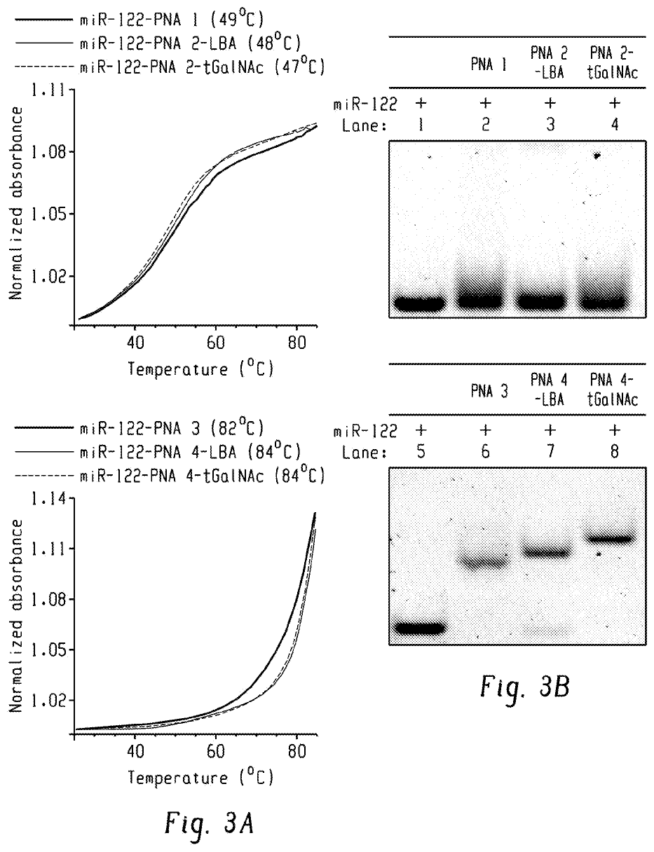

PNAs were incubated with target DNA in a 2:1 (PNA:DNA) ratio at 37° C. overnight and then samples were assessed for separation on 8% polyacrylamide gel. PNA 1, PNA 2-LBA, and PNA 2-tGalNAc showed limited target binding due to their shorter sequences designed to target the seed region of miR-122 (FIG. 3B). The full-length PNA and conjugates showed complete binding to the target DNA as seen by their retarded bands (FIG. 3B). Gel images also showed more retardation in bands for the PNA 4-LBA and PNA 4-tGalNAc due to the increased molecular weight of PNA after carbohydrate ligand conjugation. Melting curve analysis confirmed these results, where the increased melting temperature was observed for the carbohydrate ligand conjugated PNA-DNA dimers (FIG. 3A).

### Example 3. Biodistribution Studies in C57BL/6J Mice

### Biodistribution of PNAs and Ligand-PNA Conjugates

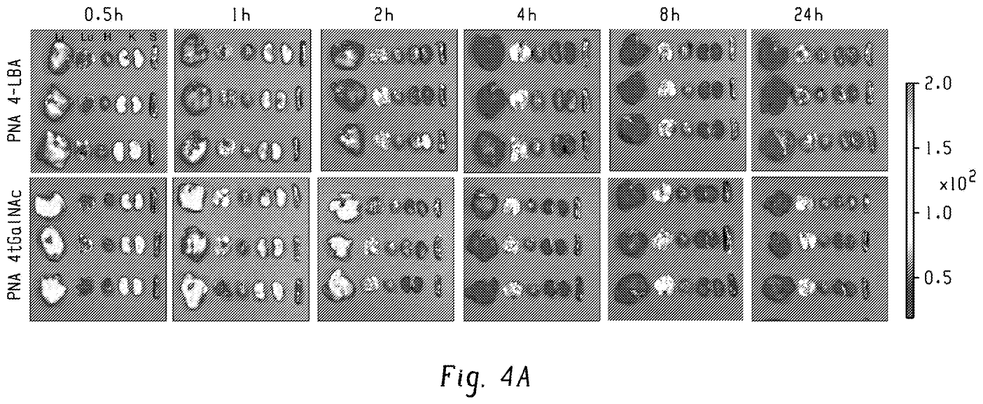

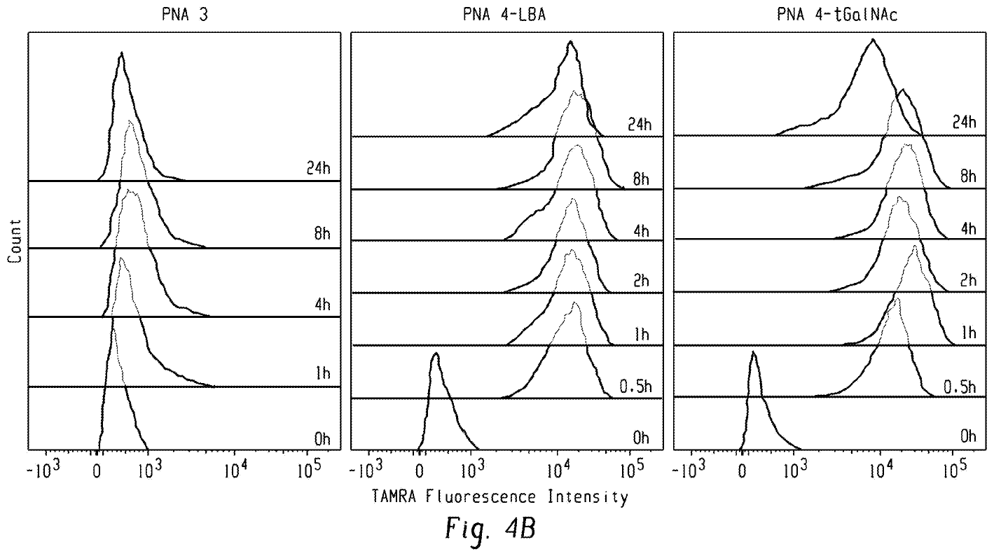

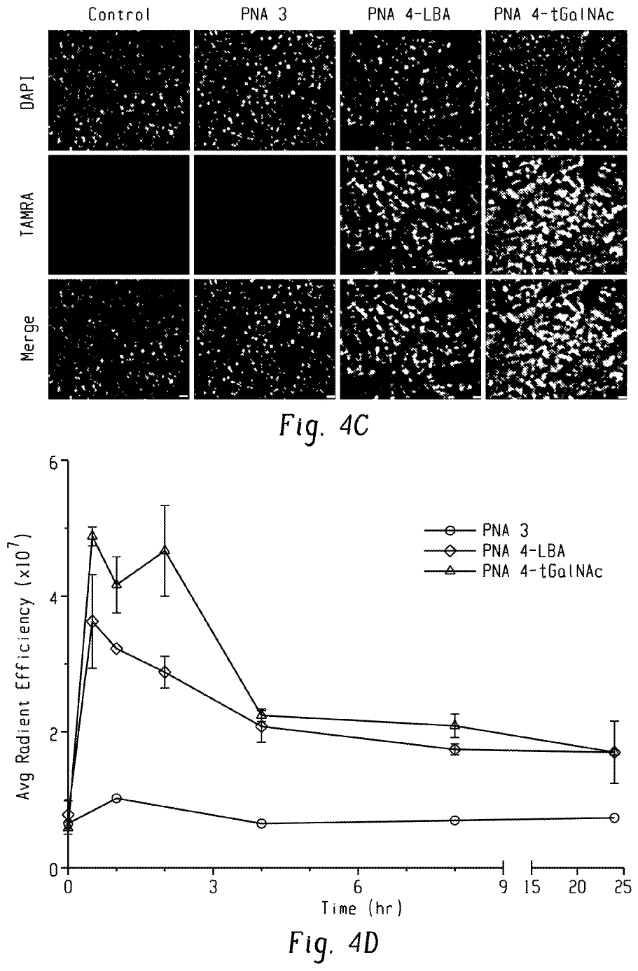

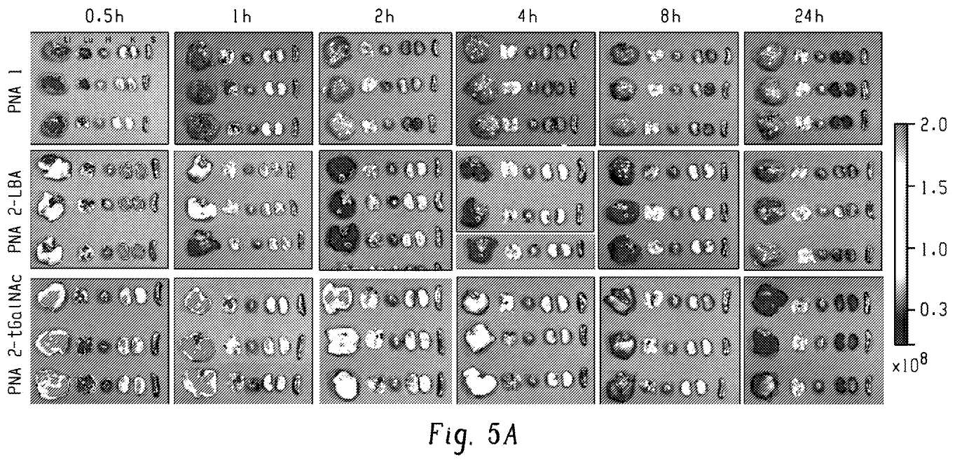

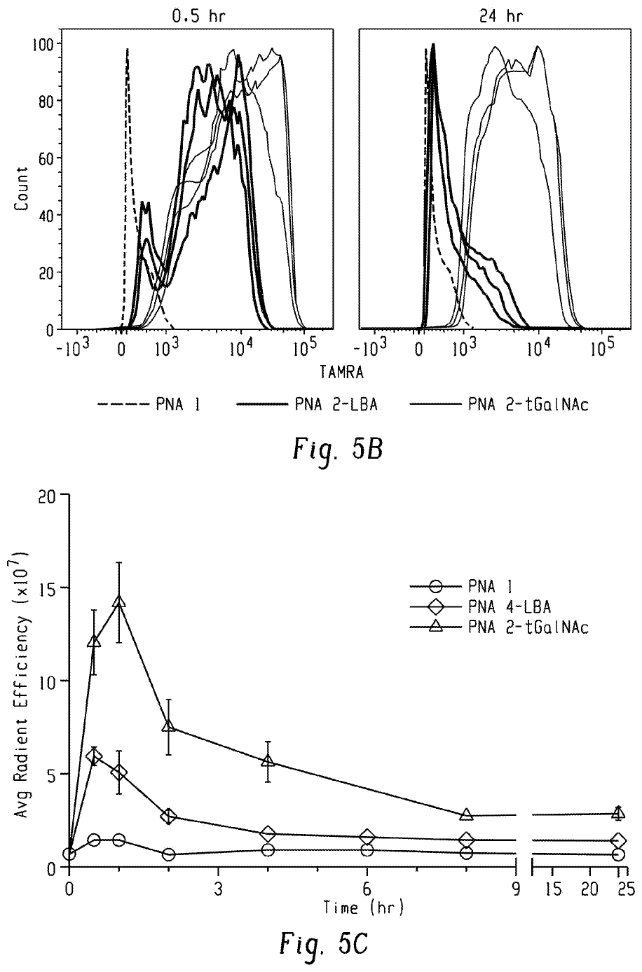

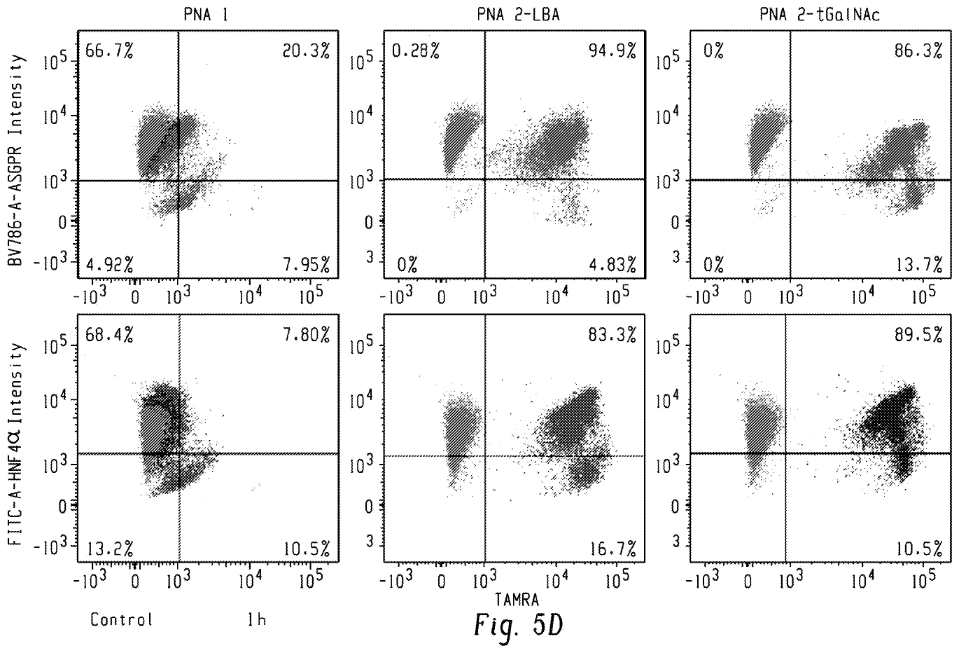

A 24 h biodistribution study after a single 125 μM, 10 ml/kg subcutaneous dose was studied in C57BL/6J mice. All PNAs were tagged with a fluorescent TAMRA (rhodamine dye) probe. Three mice each at 0.5, 1, 2, 4, 8, and 24 h were imaged for the TAMRA fluorescence on an IVIS imager and sacrificed for harvesting major internal organs. The fluorescence images of the different organs, including liver, lungs, heart, kidney, and spleen were recorded to see the relative biodistribution (FIGS. 4A and 5A). After subcutaneous administration full-length PNA (PNA 3) showed a minimal concentration in the liver, only up to 1 h. A substantial portion of the PNA 3 in the kidney from the initial time points indicated its fast elimination through renal excretion. Both PNA 4-LBA and PNA 4-tGalNAc targeted the liver very efficiently and were uniformly distributed in the liver up to 24 h (FIG. 4A). The time-dependent PNA-TAMRA fluorescence curves from isolated liver were plotted and used for calculating the relative amount of PNAs in the liver (FIG. 4D). The area under the curve (AUC) of time-liver fluorescence was calculated using GraphPad Prism software. Overall, the AUCO-24 fold change of PNA conjugates were significantly higher than PNA 3. The AUCO-24 fold change for PNA 4-LBA and PNA 4-tGalNAc was 20.34±2.53 (p**) and 25.88±4.52 (p**) respectively. PNA 4-tGalNAc showed the initial high concentration in the liver, but the change in AUCO-24 was not significant compared to PNA 4-LBA. Next, we performed the FACS analysis of liver cells after passing the liver tissue through cell strainer followed by RBC lysis. FACS data again confirmed the comparable liver targeting and retention of PNA 4-LBA and PNA 4-tGalNAc (FIG. 4B). Also, the fluorescence difference visible in IVIS images at early time points was not equally prominent in FACS analysis. Next, cryosectioning from 1 h liver samples was done for imaging TAMRA fluorescence from the tissue. The fluorescence images confirmed the uniform distribution on the PNA 4-LBA and PNA 4-tGalNAc in the liver sections (FIG. 4C).

### Biodistribution of Anti-Seed Ligand-PNA Conjugates:

Like full-length PNA (PNA 3), unconjugated short PNA 1 was also seen to be eliminated by the kidney reducing liver accumulation after 1 h of administration (FIG. 5A). Both PNA 2-LBA and PNA 2-tGalNAc delivered the PNA to the liver. Both conjugates accumulated in the liver for up to 24 h with a maximum concentration at one hour; however, concentration declined significantly with time (FIG. 5A). PNA 2-tGalNAc resulted in high liver accumulation than PNA 2-LBA throughout the study. The AUC of liver fluorescence was calculated using GraphPad Prism software, and it was significantly high for PNA-2 tGalNAc than PNA 1 and PNA 2-LBA (FIG. 5C). The AUC of PNA 2-LBA and PNA 2-tGalNAc was 9 (p*) and 28 (p****) fold higher than unconjugated PNA 1. The high liver accumulation of PNA 2-tGalNAc could be attributed by two key factors; one is higher ASGPR affinity of tGalNAc than galactose in lactobionic acid, and the other is rapid elimination of the PNA from the body. Apparently, for shorter PNAs, it seems higher ASGPR affinity of tGalNAc determines liver uptake and elimination. The organ distribution at 0.5 h also indicated that PNA 2-LBA concentration is higher in the kidney than PNA 2-tGalNAc (FIG. 5A).

Confirmation of these observations by flow cytometric analysis was performed (FIG. 5B). The histogram plots representing PNA concentration in liver cells showed good overlaps for PNA 2-LBA and PNA 2-tGalNAc at 0.5 h, but still, PNA 2-tGalNAc demonstrated higher fluorescence intensities. Conjugated PNAs showed prolonged liver retention even though there was no significant change in miR-122 levels in the liver samples.

Hepatocyte Targeting by LBA-PNA and tGalNAc-PNA Conjugates:

In in vitro studies using HepG2 cells, it was found that LBA and tGalNAc ligands showed ASGPR mediated cellular uptake. These results were confirmed following an in vivo treatment in mice. After 1 h of subcutaneous administration of PNAs and carbohydrate ligand-PNA conjugates, the mice liver was perfused and digested in situ. The collagenase-mediated liver digestion was used to separate the liver cells and the obtained liver cell suspension was enriched for hepatocytes using Percoll gradient. The obtained hepatocytes fraction was stained with ASGPR and HNF-4α (hepatocyte-specific marker) fluorescent antibodies and analyzed by flow cytometry. Quadrant 2 represents TAMRA fluorescence of PNAs, and double-positive quadrant 3 represents hepatocytes containing PNAs. The high Q3/Q2 ratio for both PNA 2-LBA and PNA 2-tGalNAc than PNA 1 confirmed the preferential accumulation of carbohydrate ligand-PNA conjugates in hepatocytes (FIG. 5D).

### Example 5. Efficacy Study

miR-122 expression is specific to the liver, and it constitutes 60-70% of the hepatocyte miRNA pool. In a healthy liver, it has a crucial role in cholesterol and fatty acid metabolism. In biodistribution studies, it was found that both LBA and tGalNAc showed excellent liver-targeted delivery of the PNAs. From the gel shift and melting curve analysis described above, it was found that the presence of a carbohydrate ligand on the 5′ end of PNA does not affect its target binding capability. Evaluating these findings for in vivo efficacy and safety was the foremost objective of this study. The levels of miR-122 were tested in liver samples from the biodistribution studies, and an excellent knockdown of miR-122 levels with PNA 4-LBA and PNA 4-tGalNAc at 4, 8, and 24 h was found. PNA 3 also showed miR-122 knockdown to some extent. But the levels of downstream targets (ALDOA, BCKDK, GYS1, NDRG3, and CUX-1) of miR-122 did not change significantly (data not provided). Either the 24 h time or/and a single dose of PNA was insufficient for knocking down the downstream targets of highly expressing miR-122 in the liver. Short PNA and conjugates did not show a significant knockdown of miR-122 due to poor binding of short PNA at the target site.

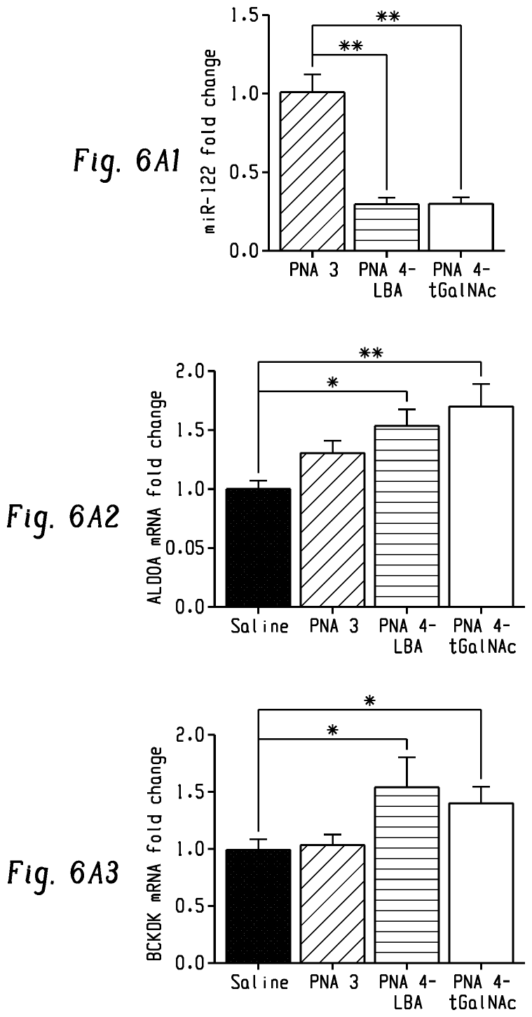

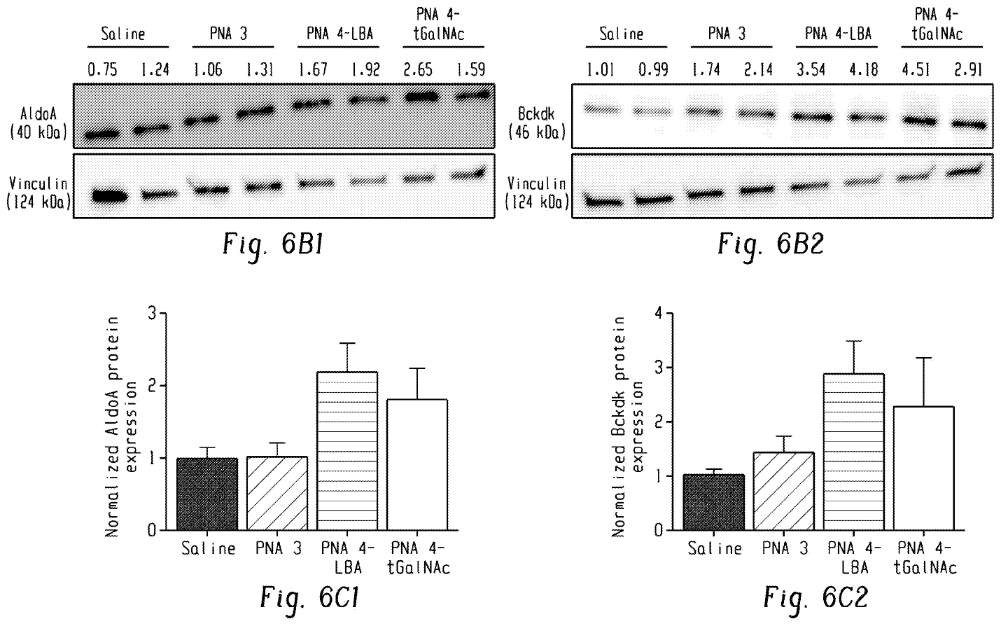

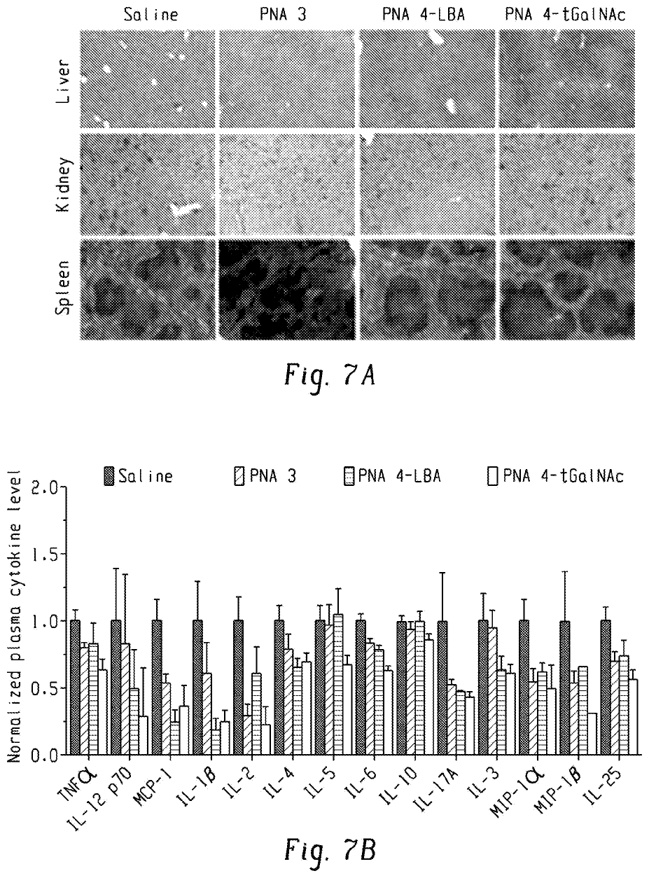

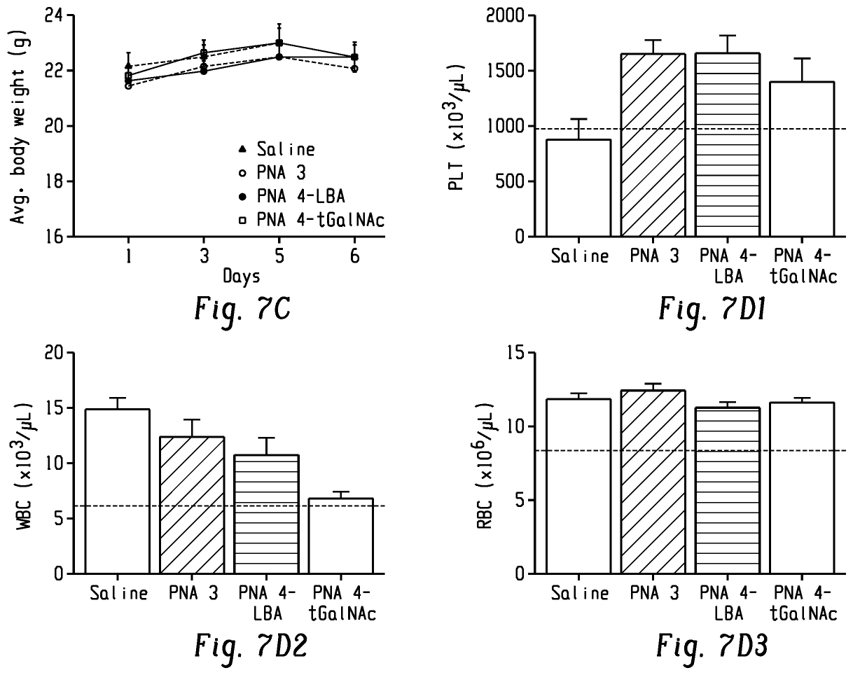

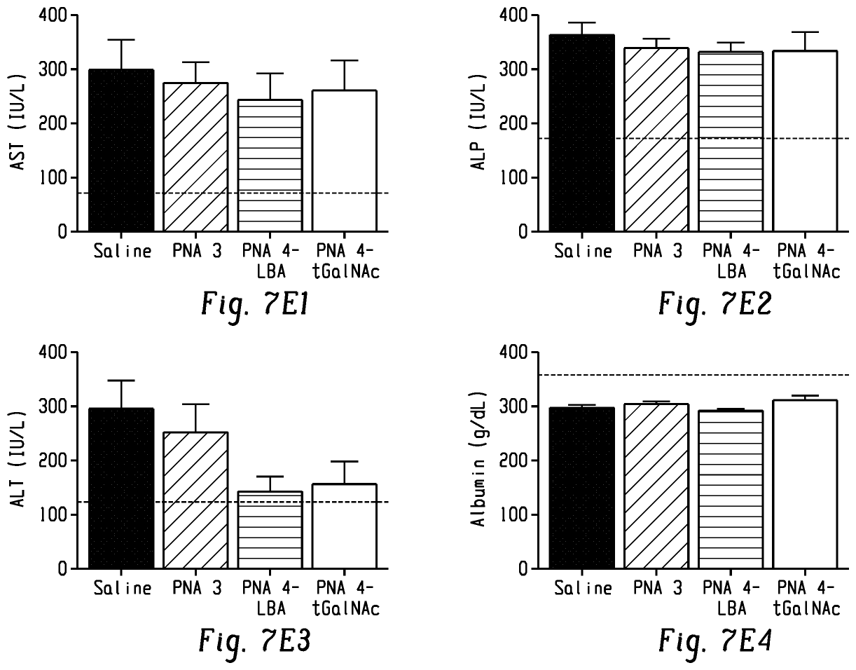

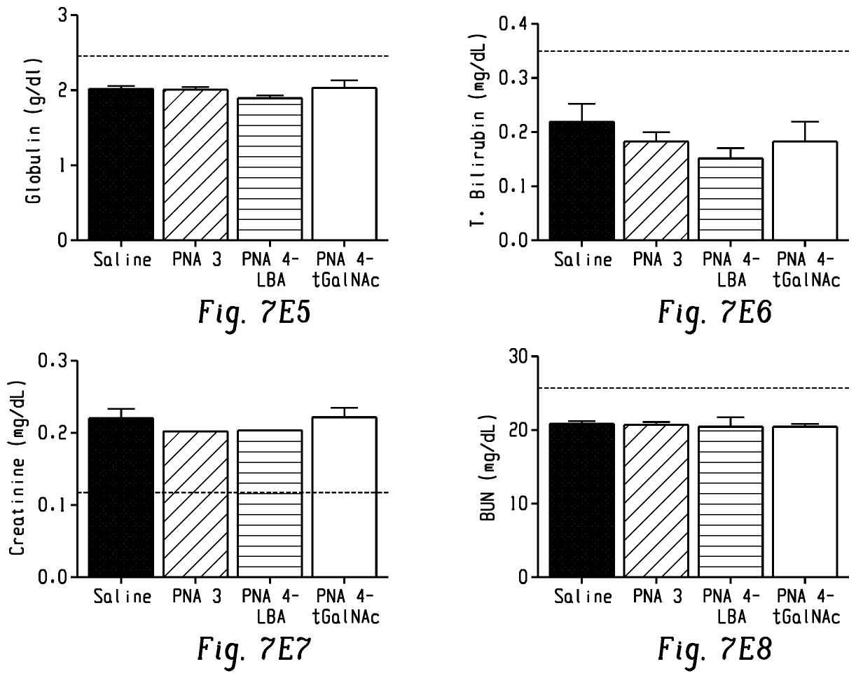

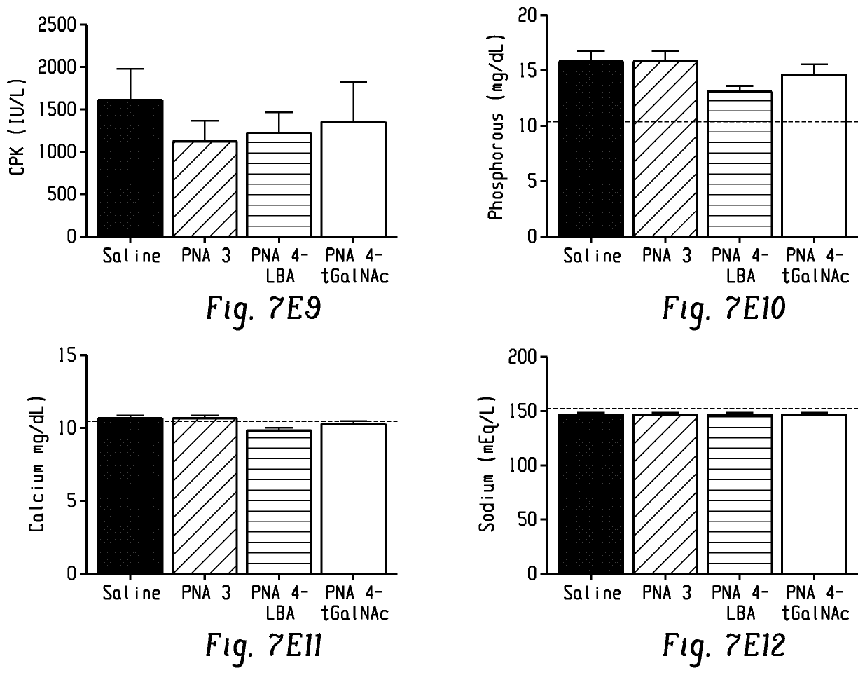

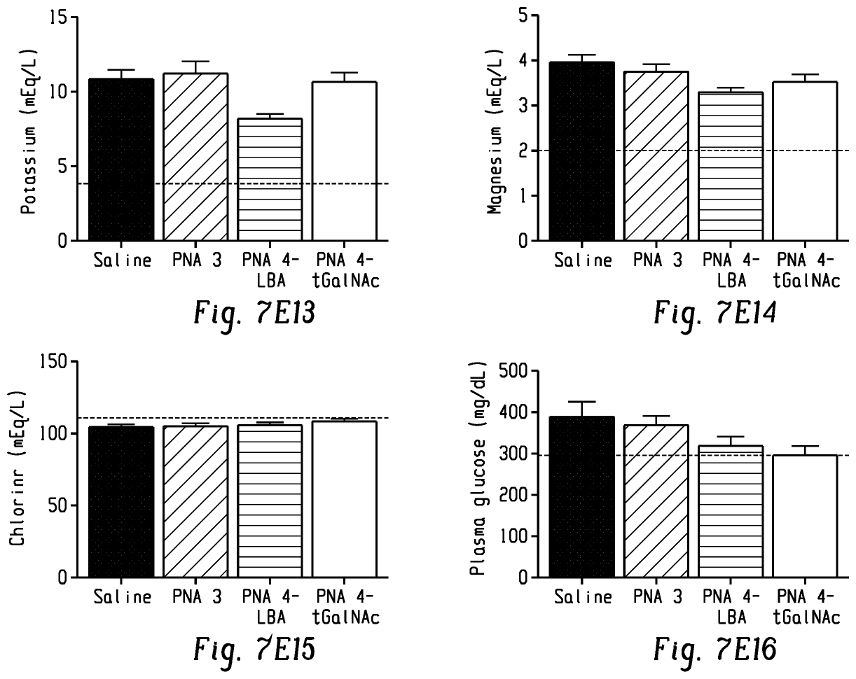

The comparison of efficacy of PNA 3, PNA 4-LBA, PNA 4-tGalNAc in a multiple-dose study was performed where a saline-treated group served as the control. For the efficacy evaluation, the levels of miR-122 and its downstream targets in liver samples by RT-PCR and western blot analysis were measured. Both PNA 4-LBA and PNA 4-tGalNAc showed up to 75% significant knockdown of miR-122 than PNA 3 (p**) (FIG. 6A). mRNA expression analysis of the downstream targets ALDOA, BCKDK, GYS1, NDRG3, and CUX-1 showed a significant increase in the mRNA levels for PNA 4-LBA and PNA 4-tGalNAc treatment (>p*) (FIG. 6A). Next, western blot analysis for AldoA and Bckdk proteins confirmed the protein upregulation after PNA 4-LBA (p*) and PNA 4-tGalNAc treatment (FIGS. 6B and 6C). PNA 3 treatment did not show a significant change in the level of downstream targets. As miR-122 is a crucial regulator of cholesterol and fatty acid metabolism, we compared the plasma levels of cholesterol and triglycerides. Plasma cholesterol and triglyceride levels of the PNA 4-LBA and PNA 4-tGalNAc group were lower than PNA 3 and saline-treated groups. The cholesterol and triglyceride levels were significantly low after PNA 4-LBA treatment (p*). PNA 4-LBA and PNA 4-tGalNAc treated groups showed lower plasma glucose levels, but the difference was not statistically significant (FIG. 7E). Furthermore, the change in the liver weight (% of body weight) was also significantly less for PNA 4-LBA and PNA 4-tGalNAc, which correlates with reduced fat storage in the liver. Overall, the changes in all the physiological parameters are not large but considering short treatment duration in healthy mice, these findings are meaningful and confirm the efficacy of PNA after targeted delivery to the liver.

### Example 6. Safety Studies

A safety assessment was performed for PNA 3, PNA 4-LBA, and PNA 4-tGalNAc in C57BL/6J mice. For safety assessment after three 5 mg/kg doses, the mice body and vital organs weight, histopathology by H&E staining (liver, kidney, and spleen), CBC analysis, comprehensive blood chemistry along with electrolyte levels and plasma levels of a panel of cytokines were evaluated. H&E staining performed on liver, kidney, and spleen tissue sections did not show any significant histological difference among treatment groups (FIG. 7A). No significant difference in the mice's body weight was observed among different treatment groups during the study (FIG. 7C). Likewise, during organ harvesting (liver, kidney, spleen, heart, and lungs), we did not find any overt sign of toxicity. The organ weight (% of body weight) did not vary significantly except for the reduced liver weight in PNA 4-LBA and PNA 4-tGalNAc treatment group. Since liver is a fat-storing organ, the observed reduced fat accumulation because of miR-122 knockdown could be one possible explanation for the present context. An elevated level of cytokines is a prime indicator for any immune response triggered from oligonucleotide treatment. Blood was collected at 24 h of the last dose for estimating plasma levels of cytokine TNFα, IL-12p70, MCP-1, IL-1β, IL-2, IL-4, IL-5, IL-6, IL-10, IL-17A, IL-3, MIP-1α, MIP-1β, IL-25 using a Luminex based assay. None of the cytokines were found to be upregulated, and there was no significant difference in cytokine levels in saline, PNA 3, PNA 4-LBA, and PNA 4-tGalNAc treatments (FIG. 7B). The inherent levels of the cytokines in our samples were lower than the calibration range of the kit. CBC analysis including RBC, WBC, platelets, and hemoglobin (data not shown) (FIG. 7D) and comprehensive blood chemistry including aspartate transaminase (AST), alkaline phosphatase (ALP), alanine transaminase (ALT), albumin, globulin, total bilirubin, creatinine, blood urea nitrogen (BUN), creatine phosphokinase (CPK), and electrolytes did not show any significant difference among various treatment groups (FIG. 7E). Electrolytes included phosphorus, calcium, sodium, potassium, magnesium, and chloride. Overall, evaluations concluded that the tested dose regimen of PNA 4-LBA and PNA 4-tGalNAc was highly safe and effective.

### Example 7. Kidney-Targeted Delivery of Short PNA

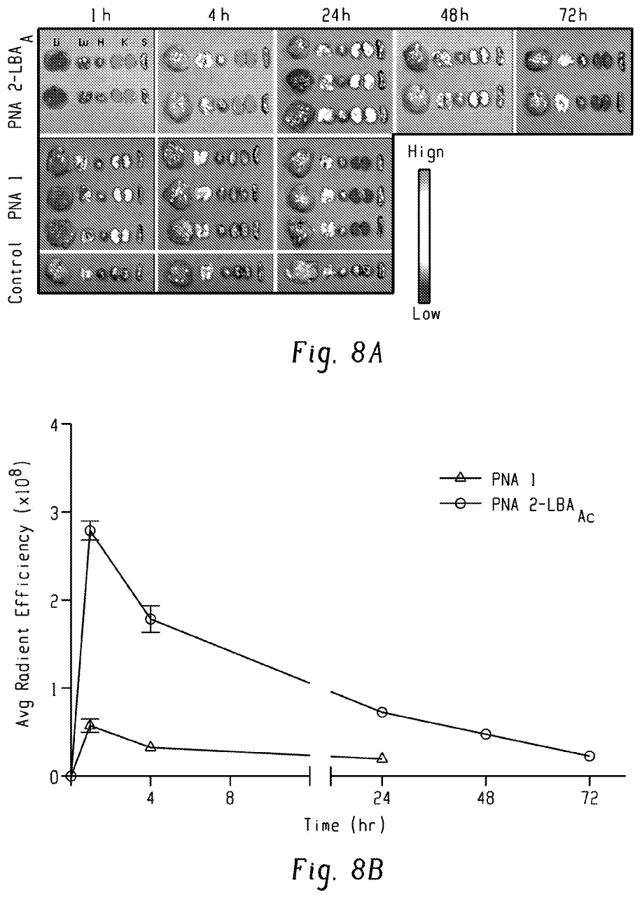

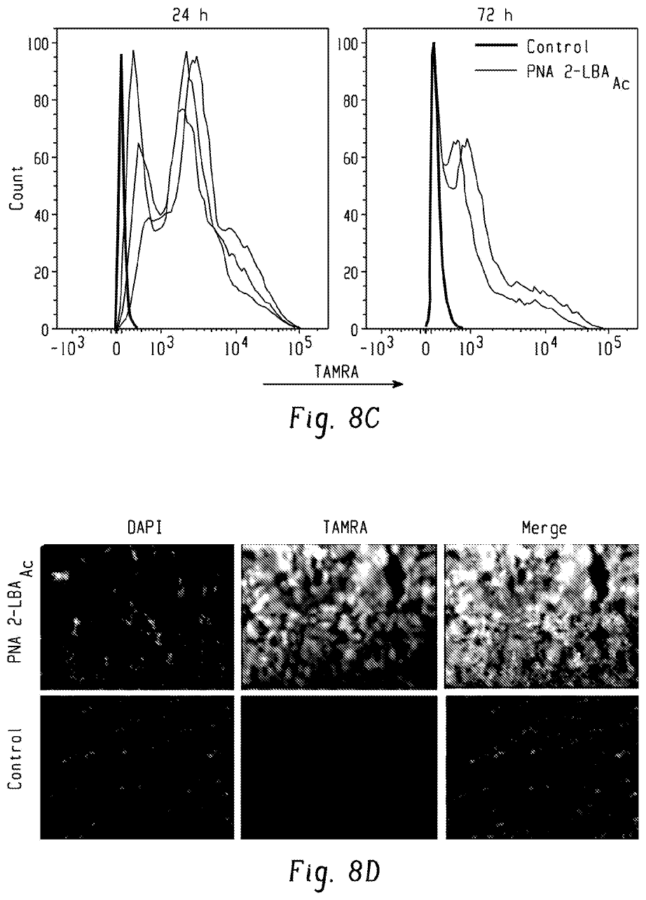

During the investigations for the functional enhancement of the LBA ligand, the acetylated LBA ligand (LBAAc) showed good potential for targeting the kidney. PNA 2-LBAAc distribution was mainly found confined to kidneys as per our biodistribution study in C57BL/6J mice (5 mg/kg, subcutaneous). Initially, at 1 h, both PNA 1 and PNA 2-LBAAc were primarily distributed in the kidney and to some extent in liver (FIG. 8A). Generally, naked/unformulated PNA has exhibited rapid kidney elimination, and we found maximum fluorescence intensity for short PNA (PNA 1) in the kidney at 30 min, indicating its elimination peak time. In comparison to PNA-1, PNA 2-LBAAc post initial distribution phase showcased maximum accumulation in the kidney (FIG. 8A). The average radiant efficiency of the TAMRA labeled PNAs from the kidney was determined at different time points (1, 4, 24, 48, and 72 h) for the quantitative analysis (FIG. 8B). Interestingly, PNA 2-LBAAc concentration in the kidney at 48 h was comparable with the elimination phase (1 h) concentration of the PNA 1. These findings prove targeting and accumulation of the PNA 2-LBAAc in the kidney.

Confirmation of PNA 2-LBAAc conjugate distribution in the kidney by flow cytometry analysis:

The kidney samples from 24 h and 72 h were passed through a 40 μm strainer, and following RBC lysis, the kidney cell suspension was fixed with 4% PFA and analyzed on a flow cytometer. Histogram plots showing the TAMRA fluorescence signal in kidney cells, confirmed the IVIS findings displaying higher retention in kidney for PNA 2-LBAAc up to 72 h as compared to PNA-1 (FIG. 8C). Both IVIS and flow cytometry analysis confirmed the kidney targeted delivery of PNA by LBAAc ligand. They kidney sections were then imaged to see the distribution of the PNA in the kidney. 10 μm thick sections from 24 h kidney samples were fixed, permeabilized, and stained with DAPI for nuclear visualization. In fluorescence imaging, a uniform distribution of PNA 2-LBAAc in the kidney (FIG. 8D) was observed. TAMRA signal intensity signifies higher tubular accumulation of the PNA 2-LBAAc.

### Kidney-Targeted Delivery of Full-Length (22Mer) PNA:

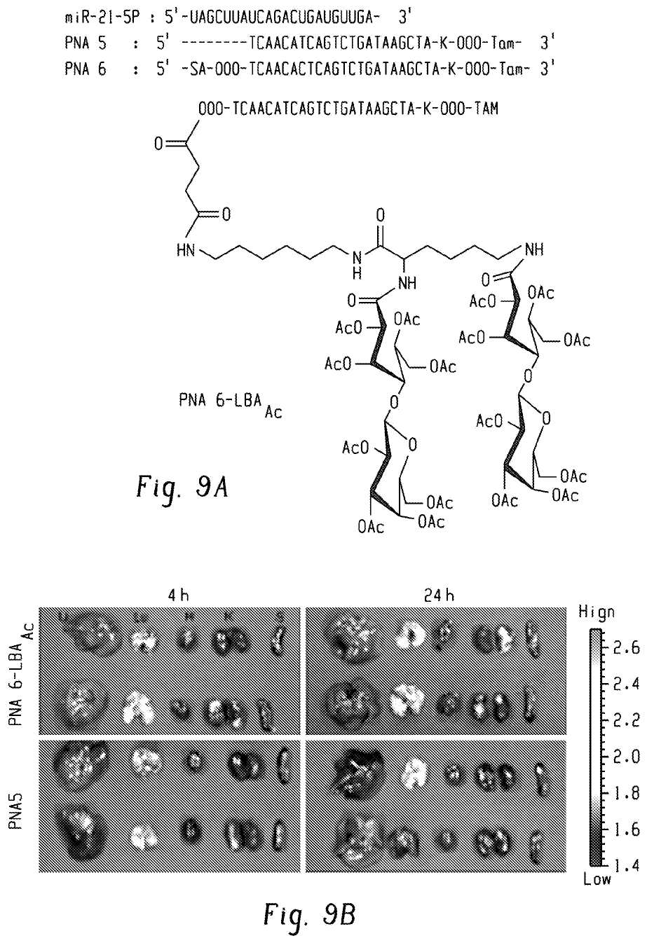

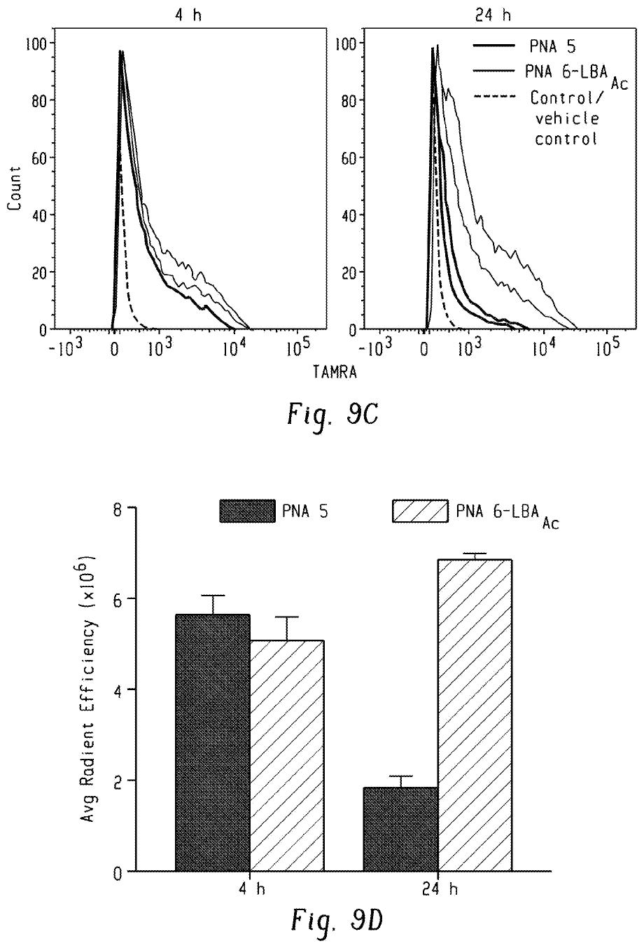

miR-21 is a kidney-specific microRNA found to be upregulated in renal fibrosis and a proven target for fibrosis treatment. Full-length (22mer) PNA oligomers were designed and synthesized namely PNA 5, PNA 6 and LBAAc conjugate (PNA 6-LBAAc) to target miR-21 (FIG. 9A). PNA 6 was conjugated with LBAAc as described in FIG. 2A.

Biodistribution evaluation of PNA 5 and PNA 6-LBAAc in C57BL/6J mice at a low subcutaneous dose of 1.5 mg/kg (1/10 molar equiv of short PNA 1&2): A lower dose biodistribution was chosen for a comprehensive validation of LBAAc for kidney targeting. From IVIS imaging, the TAMRA fluorescence signal was limited to the kidney only for both PNA 5 and PNA 6-LBAAc (FIG. 9B). At 4 h both PNA and conjugate showed a similar distribution in the kidney, and their average fluorescence intensity was comparable. At 24 h, PNA 6-LBAAc concentration increased in the kidney, whereas PNA 5 disappeared. Overall, a 4-fold increase was observed in kidney retention for PNA 6-LBAAc when compared to unconjugated PNA 5 at 24 h. (FIG. 9D). Results were confirmed by flow cytometry analysis of kidney cells. The kidney cells obtained after RBC lysis were fixed with 4% PFA and analyzed for TAMRA fluorescence. The histogram plots representing the TAMRA fluorescence from 4 and 24 h samples confirmed the elimination of PNA 5 and accumulation of PNA 6-LBAAc in the kidney cells (FIG. 9C). Overall, PNA 5 followed the usual pharmacokinetics of PNA and eliminated out fast without any tissue distribution. Whereas the PNA 6-LBAAc showed a slower biodistribution because of increased hydrophobicity of the conjugate. Possibly the Tmax for kidney accumulation of PNA 6-LBAAc is >4 h. Decisively, the higher concentration of PNA 6-LBAAc in the kidney at 24 h compared to 4 h time point against PNA 5 confirmed the kidney targeting and potential of the LBA_{Ac} ligand.

The article Dhuri K, Bechtold C, Quijano E, Pham H, Gupta A, Vikram A, Bahal R. “Antisense Oligonucleotides: An Emerging area in Drug Discovery and Development.” Journal of Clinical Medicine, 2020 is incorporated herein by reference in its entirety.

The following abbreviations are used herein.

| Abbreviation | Term |
|---|---|
| FMOC | Fluorenylmethyloxycarbonyl |
| HBTU | Hexafluorophosphate Benzotriazole |
| | Tetramethyl Uronium |
| DIEA | N,N-Diisopropylethylamine |
| tGalNAc | tris N-acetylgalactosamine |
| LBA | lactobionic acid |
| PNA | peptide nucleic acids |
| ALDOA | Aldolase A |
| BCKDK | Branched chain ketoacid |
| | dehydrogenase kinase |
| GYS1 | Glycogen [starch] synthase |
| NDRG3 | N-Myc Downstream-Regulated |
| | Gene 3 Protein |
| CUX-1 | gene for homeodomain protein |
| trioxo-miniPEG spacer | trioxo-mini polyethylene glycol spacer |
| GalNAc | N-Acetyl galactosamine |
| ASGPR | Asialglycoprotein receptors |
| DIPEA | N,N-Diisopropylethylamine |
| TFA | Trifluoroacetic acid |
| NMR Spectroscopy | Nuclear Magnetic Resonance |
| | Spectroscopy |
| miRNA | microRNA |
| MBHA | 4-Methylbenzhydrylamine resin |
| BOC | tert-butoxy carbonyl |
| TAMRA | 5-Carboxytetramethyl rhodamine |
| HPLC | High performance liquid chromatography |
| HATU | Hexafluorophosphate Azabenzotriazole |
| | Tetramethyl Uronium |
| DMF | Dimethylformamide |
| DMSO | Dimethylsulfoxide |
| NaOMe | Sodium methoxide |
| MALDI | matrix-assisted laser desorption/ionization |
| siRNA | short interfering RNA or silencing RNA |
| SYBR Gold | Dye for nucleic acid detection |
| DNA | Deoxyribonucleic acid |
| ADMET | absorption, distribution, metabolism, |
| | and excretion - toxicity |
| FACS | Fluorescence activated cell sorting |
| RBC lysis | Red Blood Cell (RBC) Lysis |
| IVIS | Non-invasive in vivo imaging |
| HNF4α | Hepatocyte nuclear factor 4 alpha |
| ANOVA | Analysis of variance |
| CBC | Complete blood count |
| H&E Stain | Hematoxylin and eosin stain |
| TNFα | Tumor necrosis factor |
| IL-12p70 | Interleukin-12p70 |
| MCP-1 | Monocyte Chemoattractant Protein-1 |
| IL-1β, IL-2, IL-4, IL-5, IL-10 | interleukins |
| MIP-1α, MIP-1β | Macrophage inflammatory protein |

Terms in this application have the following definitions.

As used herein, the compositions, methods, and articles can alternatively comprise, consist of, or consist essentially of, any appropriate components or steps herein disclosed. The compositions, methods, and articles can additionally, or alternatively, be formulated so as to be devoid, or substantially free, of any steps, components, materials, ingredients, adjuvants, or species that are otherwise not necessary to the achievement of the function or objectives of the compositions, methods, and articles. The singular forms “a,” “an,” and “the” include plural referents unless the context clearly dictates otherwise. “Or” means “and/or” unless clearly indicated otherwise by context. “Optional” or “optionally” means that the subsequently described event or circumstance may or may not occur, and that the description includes instances where the event occurs and instances where it does not.

Unless defined otherwise, technical and scientific terms used herein have the same meaning as is commonly understood by one of skill in the art to which this disclosure belongs.

As used herein, the term “hydrocarbyl” and “hydrocarbon” refer to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicyclic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxys; C₆₋₁₀ aryloxy such as phenoxy; C₁₋₆ alkylthio; C₁₋₆ or C₁₋₃ alkylsulfinyl; C₁₋₆ or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy. The indicated number of carbon atoms of a group do not include any substituents.

As used herein, a peptide nucleic acid (PNA) is an artificially synthesized polymer with a backbone comprising repeating N-(2-aminoethyl)-glycine units linked by peptide bonds. The various purine and pyrimidine bases are linked to the backbone by a methylene bridge (—CH2—) and a carbonyl group (—(C═O)—) to a nitrogen on the backbone as shown in FIG. 1. By convention, PNA is represented with the N-terminus upward (or side to the left) and the C-terminus downward (or side to the right) as in peptides. A PNA is not a peptide or a nucleic acid in the formal sense, but rather a hybrid of the two.

In some aspects, the PNA monomers forming a PNA oligomer are modified at the gamma position in the polyamide backbone (7PNAs) as illustrated below (wherein “B” is a nucleobase and “R” is a substitution at the gamma position).

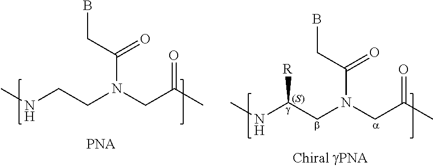

Substitution at the gamma position creates chirality and provides helical pre-organization to the PNA oligomer, yielding substantially increased binding affinity to the target RNA. Other advantageous properties can be conferred depending on the chemical nature of the specific substitution at the gamma position (the “R” group in the chiral γPNA above). The synthesis of 7PNAs is described in U.S. Pat. No. 10,221,216, incorporated herein by reference for the disclosure of γPNA and methods of synthesis of γPNA.

Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In an aspect, the PNA oligomer forming a PNA/RNA/PNA triplex is a γPNA with a tail clamp, or a γtcPNA.

Chemical modifications of the basic PNA structure are known and can be used. For example, fluorine-modified, cyclopentyl-modified, mini-peg-modified, guanidinium-modified, pyrrolidinyl-modified, and 2-aminopyridien-modified PNAs are known in the art and can be chosen for preparation of the PNA oligomer to improve cell permeability or increase RNA binding affinity. Mini-PEG-containing γ-PNAs and their methods of synthesis are described in U.S. Pat. No. 10,793,605,

The PNA oligomers can also include other positively charged moieties to increase the solubility of the PNA, for increased cell permeability, and/or to increase the affinity of the PNA for the target RNA. Commonly used positively charged moieties include the amino acids lysine and arginine, although other positively charged moieties may also be useful. Lysine and arginine residues can be added to a tcPNA linker or can be added to the carboxy or the N-terminus of a PNA oligomer strand.

Exemplary modifications to PNA include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to RNA to generate a chimeric oligomer, redesign of the backbone architecture, conjugation of PNA to DNA or RNA. These modifications improve solubility but often result in reduced binding affinity and/or sequence specificity.

Gamma-PNA modifications include serine modified, lysine modified, glutamic acid modified, or alanine modified. Particularly when the PNAs are serine gamma modified, the □PNAs target RNA more efficiently compared to the conventional full length PNAs based on their binding affinity.

Phosphorothioate analogues of DNA, RNA and OMe-RNA have sulfur in place of oxygen as one of the non-bridging ligands bonded to phosphorus.

A morpholino, also known as a morpholino oligomer and as a phosphorodiamidate morpholino oligomer (PMO), is a type of oligomer used in molecular biology to modify gene expression. Its molecular structure contains DNA bases attached to a backbone of methylenemorpholine rings linked through phosphorodiamidate groups. Morpholinos block access of other molecules to small (˜25 base) specific sequences of the base-pairing surfaces of ribonucleic acid (RNA). Morpholinos are used as research tools for reverse genetics by knocking down gene function.

Locked nucleic acid is an RNA derivative in which the ribose ring is constrained by a methylene linkage between the 2′-oxygen and the 4′-carbon. This conformation restriction increases binding affinity for complementarity sequences and provides a chemical approach for the control of gene expression and optimization of microarrays.

2′-O-methylation is a nucleoside modification of RNA, where a methyl group is added to the 2′ hydroxyl of the ribose moiety of a nucleoside, producing a methoxy group. 2′-O-methylated nucleosides are mostly found in ribosomal RNA and small nuclear RNA and occur in the functionally essential regions of the ribosome and spliceosome.

Like the 2′-OMe nucleoside modification of RNA, the 2′-O-methoxyethyl-RNA (2′-MOE) backbone provides enhanced duplex stability, significant nuclease resistance.

2′-Fluoro (2′-F) is a potent RNA analogue that possesses high RNA binding affinity and resistance to nuclease degradation.

5′-Methylcytosine is a methylated form of the DNA base cytosine (C) that regulates gene transcription and takes several other biological roles.[1] When cytosine is methylated, the DNA maintains the same sequence, but the expression of methylated genes can be altered

The G-clamp heterocycle modification, a cytosine analog that clamps on to guanine by forming an additional hydrogen bond, was rationally designed to enhance oligonucleotide/RNA hybrid affinity. PNAs containing internally-linked guanidinium moieties (GPNAs) are readily taken-up by mammalian cells, and bind to DNA and RNA with high affinity and sequence specificity.

A protecting group is a functional group that transforms a reactive functional group in an organic molecule so that it does not undergo a reaction meant for another functional group in the structure. Protecting groups are widely used in various forms in organic synthesis. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007. Adjustments to the protecting groups and formation and cleavage methods described herein may be adjusted as necessary in light of the various substituents.

A residue is used to describe any of the parts that integrate to make up a larger molecule such as a conjugate. For example, a lysine residue refers to a lysine amino acid structure integral to a conjugate covalently bonded to an alkyl diamine via the lysine carboxyl group (by an amide function) and covalently bonded by its alpha and epsilon amino groups to lactobionic acid molecules (by amide bonds) as shown in the drawings herein. A residue may also be referred to as a moiety.

RNA as used herein includes different types of RNA that serve different functions including messenger RNA, transfer RNA, ribosomal RNA, and microRNA. Micro RNA (miRNA) is involved in gene expression. miRNA is a non-coding region of mRNA that is believed to be important in the either promotion or inhibition of gene expression. These may involve small sequences of about 25 nucleotides.

A sequence of bases is a succession of bases signified by a series of a set of five different letters that indicate the order of nucleotides forming alleles within a DNA (using GACT) or RNA (GACU) molecule. By convention, sequences are usually presented from the 5′ end to the 3′ end. For DNA, the sense strand is used. Because nucleic acids are normally linear (unbranched) polymers, specifying the sequence is equivalent to defining the covalent structure of the entire molecule.

Gene expression is the process by which a genes coded information is converted into the structures present and operating in the cell. Expressed genes include those that are transcribed into mRNA and then translated into protein and those that are transcribed into RNA but not translated into protein (for example, transfer and ribosomal RNAs). miRNA is a non-coding region of mRNA that is believed to be important in the either promotion or inhibition of gene expression.

Below are brief descriptions of some gene names and micro RNAs known in the art. Other information can be found on the website https://www.genecards.org/which is incorporated herein by reference.

PKD1 gene is polycystin 1, transient receptor potential channel interacting or polycystic kidney disease 1. PKD2 gene is polycystin 2, transient receptor potential cation channel or polycystic kidney disease 2, beta-catenin is for catenin beta 1 and junction plakoglobin, glutamine synthetase is for lengsin, lens protein with glutamine synthetase domain, c-Myc for MYC binding protein, TTR for transthyretin also called prealbumin, amyloidosis type I, carpal tunnel syndrome 1, Factor VII coagulation factor VII or coagulation factor VII (serum prothrombin conversion accelerator), Eg5 is for neuronatin, PCSK9 for proprotein convertase subtilisin/kexin type 9, AAT is for apoptosis antagonizing transcription factor, TPX2 is for TPX2 microtubule nucleation factor, apoB for apolipoprotein B, SAA is for serum amyloid A1 cluster, RSV, PDGF is for platelet derived growth factor subunit A, miR-122 is for microRNA 122, miR-223 is for microRNA 223, miR-21 was among the first identified microRNAs and is located within the Vacuole Membrane Protein 1 (VMP1) locus on chromosome 17. It has been implicated in both neoplastic and non-neoplastic pathologies through many of its gene targets. Three of the main targets of miR-21 are phosphatase and tensin homolog (PTEN), Tropomyosin 1 (TPM1), and Programmed Cell Death 4 (PDCD4), miR-155 is a microRNA in humans. MiR-155 plays a role in various physiological and pathological processes involving malignant growth, viral infections, and progression of cardiovascular diseases

miR-132 is a microRNA with targets been described including mediators of neurological development, synaptic transmission, inflammation, and angiogenesis.

miR-125b is a microRNA involved in regulating NF-1B, p53, PI3K/Akt/mTOR, ErbB2, Wnt, and another signaling pathways, thereby controlling cell proliferation, differentiation, metabolism, apoptosis, drug resistance and tumor immunity.

miR-146a is a microRNA with target genes believed to be involved in the regulation of pathophysiological processes in neurological diseases, particularly the neuroinflammatory response. It is believed to play a critical role in neuroinflammation during the progression of neurological diseases.

miR-181, a microRNA precursor is a small non-coding RNA molecule transcribed as ˜70 nucleotide precursors and subsequently processed by the RNase-III type enzyme Dicer to give a ˜22 nucleotide mature product. They target and modulate protein expression by inhibiting translation and/or inducing degradation of target messenger RNAs. This new class of genes has recently been shown to play a central role in malignant transformation. miRNA are downregulated in many tumors and thus appear to function as tumor suppressor genes. The mature products miR-181a, miR-181b, miR-181c or miR-181d are thought to have regulatory roles at posttranscriptional level, through complementarity to target mRNAs.

let-7 The lethal-7 (let-7) gene was first discovered in the nematode as a key developmental regulator and became one of the first two known microRNAs (the other one is lin-4). Expression of let-7 members is controlled by MYC binding to their promoters. Let-7 has been demonstrated to be a direct regulator of RAS expression in human cells. Numerous reports have shown that the expression levels of let-7 are frequently low, and the chromosomal clusters of let-7 are often deleted in many cancers. Let-7 is expressed at higher levels in more differentiated tumors, which also have lower levels of activated oncogenes such as RAS and HMGA2. Therefore, expression levels of let-7 could be prognostic markers in several cancers associated with differentiation stages.

miR-34a is a micro RNA that has been detected to be dysregulated in various cancers, and also is the first miRNA that demonstrated to be directly regulated by the tumor suppressor p53. The miR-34 family is known to inhibit tumorigenesis. The expression of miR-34 family relies on endogenous expression or mimics transfection.

miR-805 is a microRNA reported to be down-regulated in LPS-treated macrophages. miR-690, a micro RNA is highly expressed in M2 polarized bone marrow-derived macrophages exosomes and is reported to function as an insulin sensitizer both in vivo and in vitro.

miR-134 is a brain-specific microRNA; it is reported to be localised specifically in hippocampal neurons and may indirectly regulate synaptic development through antisense pairing with LIMK1 mRNA. In the human brain, SIRT1 is thought to mediate CREB protein through miR-134, giving the microRNA a role in higher brain functions such a memory formation.

Polycystic kidney disease (PKD) is an inherited disorder in which clusters of cysts develop primarily within the kidneys, causing your kidneys to enlarge and lose function over time. Cysts are noncancerous round sacs containing fluid. The cysts vary in size, and they can grow very large. Having many cysts or large cysts can damage the kidneys.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While typical aspects have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

1. A genetic sequence-carbohydrate conjugate of a formula: 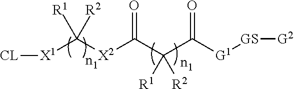
wherein
GS is a genetic sequence, the genetic sequence having a 3′ end and a 5′ end,
R₁ and R₂ are each independently H or a substituted or unsubstituted C₁ to C₆ alkyl,
X¹ is O, NR³, C═O, or C(R³)₂ where R³ is H or a substituted or unsubstituted C₁ to C₆ alkyl,
X² is O, NR³, or C(R³)₂ where R³ is H or a substituted or unsubstituted C₁ to C₆ alkyl,
G¹ is a direct bond or a group linking the PNA to the conjugate,
G² is H or a functional moiety,
CL is a carbohydrate ligand comprising 2 to 16 carbohydrate residues derived from a monosaccharide, a disaccharide, a trisaccharide, or a tetrasaccharide, preferably wherein CL comprises a carbohydrate residue derived from a monosaccharide or a disaccharide,
optionally wherein the carbohydrate ligand is fully or partially acylated on a hydroxy or amino group thereof with a C₂ to C₁₅ acyl group, preferably wherein the carbohydrate ligand is fully or partially acetylated on a hydroxy or amino group thereof,
n¹ is 1 to 20, and
n² is 0 to 20.

2. The conjugate of claim 1, wherein the genetic sequence comprises a PNA, mRNA, or siRNA that comprises a chemically modified nucleotide, for example a locked nucleic acid (LNA), phosphorothioate (PS), phosphorodiamidate morpholino (PMO), 2′-O-methyl (2′-O-Me), G-clamp, 2′-O-methoxyethyl (2′-O-MOE), siRNA, 2′-fluoro (2′F), 5′-methylcytosine, or a combination thereof.

3. The conjugate of claim 1, wherein the genetic sequence is effective to target asialoglycoprotein receptor (ASGPR) on hepatocytes, or wherein the PNA is effective to target kidney cells.

4. The conjugate of claim 1, for modulating a target gene, a target mRNA, a microRNA, or a non-coding RNA.

5. The conjugate of claim 4, wherein the target gene or RNA is PKD1, PKD2, beta-catenin, glutamine synthetase, c-Myc, TTR, Factor VII, Eg5, PCSK9, AAT, TPX2, apoB, SAA, RSV, PDGF, miR-122, miR-223, miR-21, miR-155, miR-132, miR-125b, miR-146a, miR-181, let-7, miR-34a, miR-805, miR-690, miR-134, miR-494, miR-202-5p, or miR-192.

6. The conjugate of claim 1, wherein G¹ has 1 to 20 carbon atoms, or wherein G¹ is functional moiety linking the PNA to the conjugate, for example wherein G¹ is a residue of a polyethylene glycol, a polypropylene glycol, or a polyethylene-propylene glycol such a residue of a trioxo-mini polyethylene glycol (PEG)chain.

7. The conjugate of claim 1, wherein G² has 1 to 20 carbon atoms, or wherein G² is a functional moiety linking the PNA to the conjugate, for example wherein G² is a residue of a polyethylene glycol, a polypropylene glycol, or a polyethylene-propylene glycol such as a trioxo-mini-PEG chain.

8. The conjugate of claim 1, wherein the carbohydrate ligand further comprises a linker for attachment to X¹.

9. The conjugate of claim 1, wherein the carbohydrate residue is a fully or partially acylated carbohydrate residue wherein the acyl groups have 2 to 15 carbon atoms, for example a fully or partially acetylated carbohydrate residue.

10. The conjugate of claim 1, wherein a functional moiety on the conjugate further comprises a linker for attachment to kielin, tolvaptan, nintedanib, paclitaxel, bleomycin, cyclosporin, cisplatin, romidepsin, doxorubicin, docetaxel, danunorubicin, vincristine, methotrexate, cyclophosphamide, venetoclax, hydroxyurea, mercaptopurine, prednisolone, cytarabine, or pirfenidone.

11. The conjugate of claim 1, for modulating a target gene, mRNA, microRNAs, a non-coding RNA, a DNA, a hormone, a cellular protein, or an enzyme.

12. The conjugate of claim 1, wherein the target gene or RNA is PKD1, PKD2, GPX1, GPX4, CYP11B2, ERCC4, ERCC2, GSTO1, GSTO2, UMOD, MGP, GLO1, SLC7A9, SHROOM3, VEGFA, APOL1, MYH9, miR-21, miR-17, MiR-10, miR-192, miR-216a and miR-217, miR-192, miR-377 miR-200c, miR-141, miR-205 and miR-192.

13. The conjugate of claim 1, wherein the carbohydrate residue is derived from N-acetylgalactosamine (GalNAc), galactose, lactobionic acid or an acetylated ester thereof, preferably wherein the carbohydrate residue is a fully or partially acetylated product of N-acetylgalactosamine (GalNAc), galactose, or lactobionic acid.

14. The conjugate of claim 1, of a formula 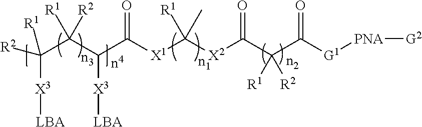
wherein
LBA is a lactobionic acid residue,
X¹ is NR³, O, or C(R³)₂ where R³ is H or a substituted or unsubstituted C₁ to C₆ alkyl,
each X³ is independently O, NR³, or C(R³)₂ where R³ is H or a substituted or unsubstituted C₁ to C₆ alkyl,
n₃ is 0 to 20, and
n₄ is 1 to 8.

15. The conjugate of claim 13, wherein
G¹ is a group linking the PNA to the conjugate,
R¹ and R² are each H,
X¹, X², and X³ are each NH, and
n₁=6, n₂=2, and n₃=4.

16. The conjugate of claim 13, wherein
R¹ and R² are each H,
n₃=4, and
the PNA is linked at the 5′ end to the conjugate.

17. The conjugate of claim 13, of a formula: 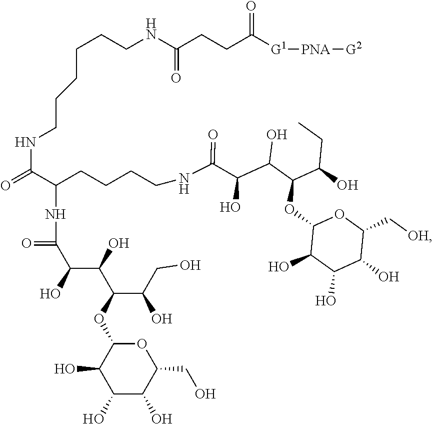
preferably wherein G¹ is a functional moiety linking the PNA to the conjugate and G² is a functional moiety.

18. The conjugate of claim 13, wherein the lactobionic acid residue is fully or partially acylated with an acyl group having from 2 to 15 carbon atoms, preferably wherein the lactobionic acid residue is fully or partially acetylated.

19. The conjugate of claim 1, of a formula 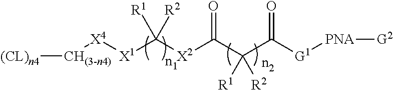
wherein
X¹ is C═O or C(R³)₂ where R³ is H or a substituted or unsubstituted C₁ to C₆ alkyl,
X⁴ is O, NR³, or C(R³)₂ where R³ is H or a substituted or unsubstituted C₁ to C₆ alkyl,
CL is a carbohydrate residue linked to CH by a 1 to 30 atom linker chain comprising a substituted or unsubstituted C₁ to C₁₂ alkyl or a C₆ to C₁₂ aryl comprising an amide, ester, or ether group, and
n₄ is 2 or 3.

20. The conjugate of claim 18, wherein the carbohydrate residue is a fully or partially acylated carbohydrate residue wherein the acyl groups have 2 to 15 carbon atoms, for example a fully or partially acetylated carbohydrate residue.

21. The conjugate of claim 19, wherein the carbohydrate residue is derived from N-acetylgalactosamine (GalNAc) or from a fully or partially acylated acetylgalactosamine.

22. The conjugate of claim 1, formulated with a polymer, lipid, protein, or other pharmaceutical excipient for organ-specific delivery.

23. A method of conjugating a genetic sequence to a carbohydrate ligand to provide the genetic sequence-carbohydrate conjugate of claim 1, wherein X² is O or NR³, the method comprising:
functionalizing the genetic sequence to provide free —COOH functionality; and
forming a bond between the free —COOH functionality of modified genetic sequence and Y² of a compound of a formula 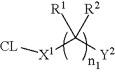
wherein Y² is —NHR³ or —OH.

24. The method claim 23, wherein the conjugating is by solution-phase synthesis, solid-phase synthesis, or a combination thereof.

25. The method of claim 22, further comprising modifying the genetic sequence with a precursor of G¹, G², or a combination thereof, before functionalizing the genetic sequence.

26. A method of conjugating a genetic sequence to a lactobionic acid-backbone ligand to provide the genetic sequence-lactobionic acid conjugate of claim 13, wherein X¹ and X² is each independently O or NR³, the method comprising:
functionalizing the genetic sequence to provide free —COOH functionality; and
forming a bond between the free —COOH functionality of modified genetic sequence and Y² of a formula 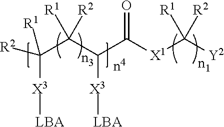
wherein Y² is —NHR³ or —OH.

27. The method of claim 26, further comprising
reacting lactobionic acid with a backbone of the formula 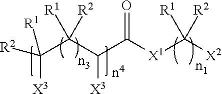
wherein
X³ is an —OH or NHR³, and
X² is a protected O or protected NHR³.

28. A method for reducing expression of a targeted RNA involved in a health disorder in a subject, the method comprising:
providing to a cell of the subject in vivo or ex vivo the genetic sequence-lactobionic acid conjugate according to claim 1, wherein the binding of the PNA of the conjugate to the targeted RNA reduces expression of the targeted RNA.

29. The method of claim 27, wherein the targeted RNA is a microRNA.

30. A method for targeting DNA and gene editing in a health disorder in a subject, the method comprising:
providing to a cell of the subject in vivo or ex vivo a genetic sequence-carbohydrate conjugate according to claim 1, wherein the DNA of the conjugate targeted to the cell modulates expression of a gene.

31. The method of claim 27, targeting liver or kidney cells to regulate expression of cellular nucleic acid function to a subject in need thereof, comprising administering to the subject the genetic sequence-carbohydrate conjugate of claim 1.

32. The method of claim 27, wherein the cell is a cancer cell.

33. The method of claim 1, wherein the PNA comprises a kidney-specific microRNA, miR-21.

34. The method of claim 31, for the treatment of renal fibrosis and polycystic kidney disease.

35. A pharmaceutical composition comprising the conjugate of claim 1, and a pharmaceutical excipient.
